(19) United States
(12) Patent Application Publication
(10) Pub. No.: US 2026/0274828 A1
(43) Pub. Date: Sep. 17, 2026

(54) BROAD SPECTRUM ANTIFUNGAL KETO-ALKYL-PYRIDINIUM COMPOUNDS

(71) Applicants: UNIVERSITY OF IOWA RESEARCH FOUNDATION, Iowa City, IA (US); BOARD OF TRUSTEES OF NORTHERN ILLINOIS UNIVERSITY, Dekalb, IL (US)

(72) Inventors: Damian Krysan, Iowa City, IA (US); Timothy Hagen, Dekalb, IL (US); Sarah Beattie, Iowa City, IA (US)

(21) Appl. No.: 19/167,240

(22) PCT Filed: Mar. 21, 2024
(86) PCT No.: PCT/US2024/020948

§ 371 Date: Sep. 19, 2025

Related U.S. Application Data
(60) Provisional application No. 63/453,660, filed on Mar. 21, 2023.

(51) Int. Cl. C07D 405/06; A01N 43/40; A01P 1/00; A61K 31/4425; A61K 31/443; A61L 15/44; A61P 31/10; C07D 213/73

(52) U.S. Cl. CPC C07D 405/06; A01N 43/40; A01P 1/00; A61K 31/4425; A61K 31/443; A61L 15/44; A61P 31/10; C07D 213/73; A61L 2300/406

(57) ABSTRACT

The disclosure relates to a class of antifungal compounds that have broad-spectrum activity.

### CLAIM OF PRIORITY

This patent application claims the benefit of priority to U.S. Provisional Application Ser. No. 63/453,660, filed Mar. 21, 2023, which is incorporated by reference herein in its entirety.

### STATEMENT OF GOVERNMENT RIGHTS

This invention was made with government support under grant 1 F32 AI145160 awarded by the National Institutes of Health. The government has certain rights in the invention.

### BACKGROUND

The effective treatment of human fungal infections is reliant on a relatively small set of structurally and mechanistically distinct drugs: azoles, polyenes, and echinocandin/triterpenoid glucan synthase inhibitors (Wiederhold, 2022). As has been extensively discussed in the literature, this limited antifungal pharmacopeia must be expanded to meet the challenging clinical needs of patients in the 21st century (Perfect et al., 2022). Two specific examples where current antifungal therapies are relatively ineffective are: 1) infections involving biofilm on medical devices and mucosal surfaces (Taff et al., 2013) and 2) decolonization of Candida auris from skin (Kean & Ramage, 2019). As with antibacterial agents, fungal biofilms are highly resistant to the currently used antifungal drugs (Taff et al., 2013). Consequently, removal of Candida infected medical devices such as vascular catheters and prosthetic joints is the standard of care (Pappas et al., 2016), whereas devices infected with some species of bacteria are frequently salvaged with antibiotic therapy alone (Mermel et al., 2009).

C. auris is an emerging fungal pathogen with many unusual characteristics compared to other Candida species (Taff et al., 2013). For example, it is readily transmitted from one patient to another in the hospital setting (Horton & Nett, 2020). This is relatively uncommon for other fungal pathogens and is likely due to its ability to persistently colonize patient's skin as well as the inanimate surfaces in hospitals and long-term care facilities. Standard biocides used to decolonize microbes (e.g., methicillin-resistant Staphylococcus aureus) from patient's skin such as chlorhexidine have relatively poor activity against C. auris (Johnson et al., 2021). Furthermore, C. auris is frequently resistant to at least one of the three major classes of antifungal drugs (Rybak et al., 2022) and pan-resistant isolates have been reported with increasing frequency (Jacobs et al., 2022). As such, C. auris is a pernicious problem that can result in therapeutically intractable infections.

One approach to treating surface-associated fungal infections such as biofilms and skin colonization is to identify molecules that are mechanistically distinct from those used to treat systemic infections and are specifically developed for that purpose. Such molecules could be imagined to display the general properties of traditional antiseptics but with activity optimized for specific organisms or improved safety profile. Here, we describe a set of novel N-keto alkyl pyridinium ions with broad spectrum antifungal activity against both planktonic and biofilm phase fungi.

Pyridinium-based anti-septic/anti-infective molecules have been studied and developed for topical and, in some instances, possible systemic use. Molecules in this class have been explored for antifungal applications (Chen et al., 2010; Shen et al., 2020). For example, the long chain N-alkylated pyridinium cetylpyridinium chloride, a component of commercial mouthwashes, has in vitro activity against Candida spp with minimum inhibitory concentrations (MIC) of 2-6 μg/mL in modified CLSI conditions (Edlind et al., 2005). A recent repurposing screen found that the anti-parasitic pyridinium-class drug pyrvinium pamoate is active against C. auris (MIC 2 μg/mL, Simm et al., 2022). Bis(pyridinium) molecules with variously sized alkyl or aryl linkers have also been shown to have antifungal activity (Chen et al., 2010; Obando et al., 2018). One study showed that the bis(pyridinium) compounds induced less red blood cell lysis than similar bis(quaternary-ammonium) compounds (Obando et al., 2018).

### SUMMARY

The disclosure provides a class of antifungal compounds that have broad-spectrum activity, e.g., the compounds kill a variety of fungal cells. The distinction between compounds that kill fungal cells versus those that stop fungal growth is important because patients who develop fungal infections are often immune deficient and do not have adequate immune activity to help resolve infections. Furthermore, current antifungal drugs have limited spectrum and are not active against some fungal pathogens particularly molds which cause aggressive and hard to treat infections. The present compounds have excellent activity against fungal biofilms and maybe employed for the topical treatment of biofilm-related fungal diseases to help prevent morbidity and mortality associated with these infections. In particular, the compounds have been tested for efficacy against several mold and yeast species in vitro and in two ex vivo models of fungal disease. The toxicity in red blood cells and a liver cell line, as well as initial in vitro and in vivo PK studies in mice, of the compounds is conducted.

Exemplary compounds of the disclosure are mono-pyridinium ions with aryl-alkyl ketone substituents at the N atom of the pyridinium ring (FIG. 1A). The molecules are comparable in activity to the commercially used, cetylpyridinium chloride and pyrvinium pamoate and have activity in an in vivo rat model of C. albicans vascular catheter infection as well as in an ex vivo model of C. auris skin colonization.

Thus, the disclosure provides a compound having a structure according to Formula I:

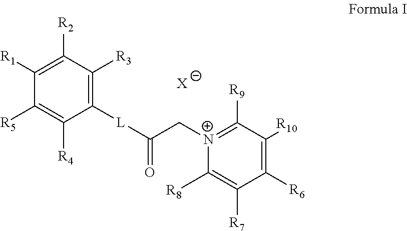

wherein
R1 and R2 are each independently H, alkyl, OH, SH, alkoxy, alkylthio, aryloxy, arylthio, alkyl, aryl, arylmethyl, arylmethoxy, F, Cl, Br, I, CF₃, CF₂H, CN, NO₂, NH₂, NH(alkyl), NH(aryl), N(alkyl)₂, NH(CO)(alkyl), or NH(CO)(aryl), or R1 and R2 taken together with the atoms to which they are attached form a 1,3-dioxolane ring and have the structure

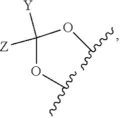

wherein Y and Z are each independently H, alkyl, or aryl;
R3 is H, alkyl, OH, SH, alkoxy, alkylthio, aryloxy, arylthio, alkyl, aryl, arylmethyl, arylmethoxy, F, Cl, Br, I, CF₃, CF₂H, CN, NO₂, NH₂, NH(alkyl), NH(aryl), N(alkyl)₂, NH(CO)(alkyl), or NH(CO)(aryl);
R4 and R5 are each independent H, alkyl, OH, SH, alkoxy, alkylthio, aryloxy, arylthio, alkyl, aryl, arylmethyl, arylmethoxy, F, Cl, Br, I, CF₃, CF₂H, CN, NO₂, NH₂, NH(alkyl), NH(aryl), N(alkyl)₂, NH(CO)(alkyl), or NH(CO)(aryl), or R4 and R5 taken together with the atoms to which they are attached form a fused aromatic ring;
L is a bond or an aromatic ring;
R6 is H, alkyl, OH, SH, alkoxy, alkylthio, aryloxy, arylthio, alkyl, aryl, arylmethyl, arylmethoxy, F, Cl, Br, I, CF₃, CF₂H, CN, NO₂, NH₂, NH(alkyl), NH(aryl), N(alkyl)₂, NH(CO)(alkyl), or NH(CO)(aryl);
R7, R8, R9, and R10 are each independently H, alkyl, OH, SH, alkoxy, alkylthio, aryloxy, arylthio, alkyl, aryl, arylmethyl, arylmethoxy, F, Cl, Br, I, CF₃, CF₂H, CN, NO₂, NH₂, NH(alkyl), NH(aryl), N(alkyl)₂, NH(CO)(alkyl), or NH(CO)(aryl); and
X is a pharmaceutically acceptable conjugate base.

In one embodiment, at least one of R1 and R2 are aryloxy, or R1 and R2 taken together with the atoms to which they are attached form a 1,3-dioxolane ring and have the structure:

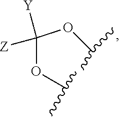

wherein Y and Z are each independently H, alkyl, or aryl. In one embodiment, Y and Z are each aryl. In one embodiment, L is phenylene. In one embodiment, L is a bond. In one embodiment, R6 is H, NH₂, NH(alkyl), NH(aryl), N(alkyl)₂, NH(CO)(alkyl), or NH(CO)(aryl). In one embodiment, R6 is H. In one embodiment, R6 is NH₂. In one embodiment, R6 is NH(CO)(alkyl). In one embodiment, R3, R4, and R5 are each H. In one embodiment, R7, R8, R9, and R10 are each H. In one embodiment, R4 and R5 taken together with the atoms to which they are attached have the structure:

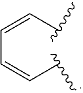 [SMILES: */C=C\C=C/*]

In one embodiment, the compound has a structure according to Formula II:

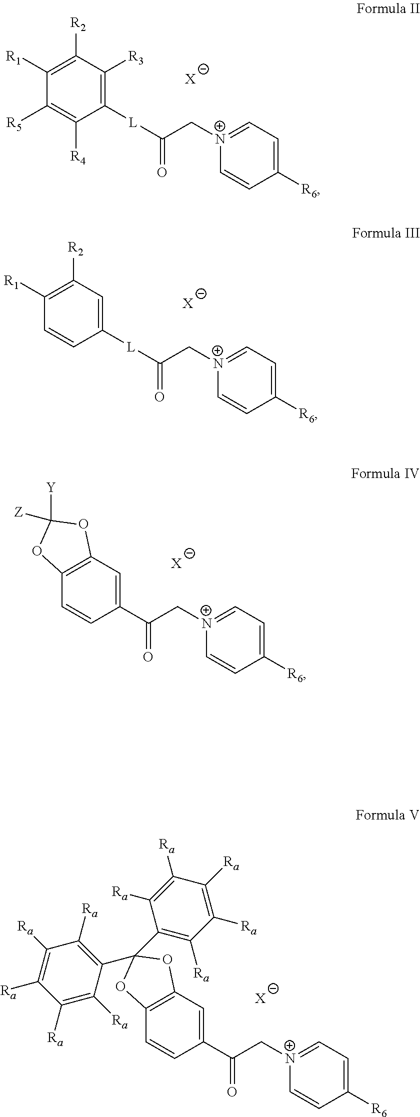

wherein Ra is H, alkyl, OH, SH, alkoxy, alkylthio, aryloxy, arylthio, alkyl, aryl, arylmethyl, arylmethoxy, F, Cl, Br, I, CF₃, CF₂H, CN, or NO₂, Formula VI:

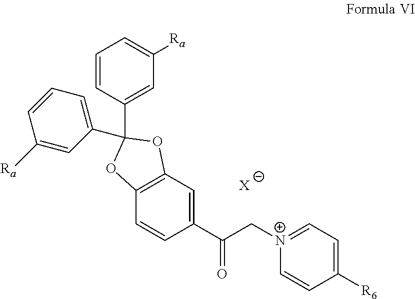

wherein Ra is H, alkyl, OH, SH, alkoxy, alkylthio, aryloxy, arylthio, alkyl, aryl, arylmethyl, arylmethoxy, F, Cl, Br, I, CF₃, CF₂H, CN, or NO₂, or Formula VII:

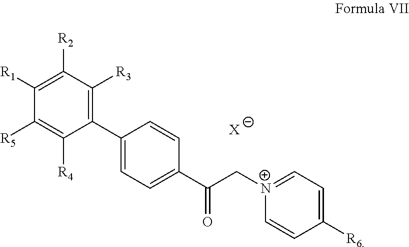

In one embodiment, X is a Cl or Br.

Also provided is a compound having the structure:

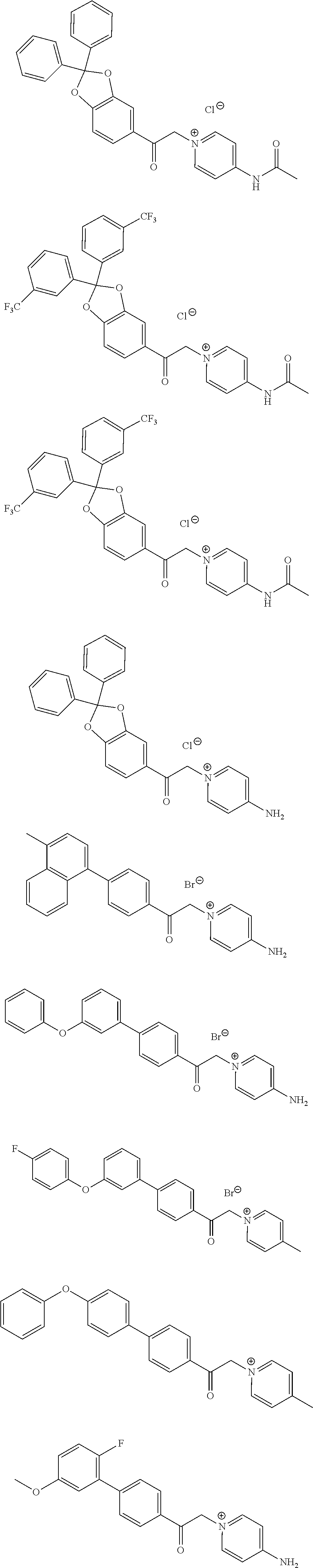

Further provided is pharmaceutical composition comprising one or more of the compounds and a pharmaceutically acceptable carrier. The compounds or compositions having one or more of the compounds may be employed, for example, in a method of killing a fungal cell, comprising contacting the fungal cell with the compound or composition. In one embodiment, a method for topical treatment of fungal infection is provided comprising topically administering the compound or composition to an area of fungal infection in a subject. In one embodiment, a method of destroying a fungal biofilm or hyphae, comprising contacting the compound or the composition with the fungal biofilm or hyphae. Also provided is an anti-infective catheter, comprising a catheter having a lumen that contains the compound or the composition.

### BRIEF DESCRIPTION OF THE FIGURES

FIG. 1. Ketoalkylpyridinium compounds, e.g., with aryl substitutions, have tunable antifungal activity. The pharmacophore and R₁ substitutions for compounds A, B and C with minimum inhibitory concentrations (MIC; μg/mL) for C. albicans SC5314, C. auris 0381, and A. fumigatus CEA10.

FIG. 2. KAPs are active against fungal biofilms. Metabolic activity of established C. albicans SC5314 (A) or C. auris 0381 (B) biofilms treated with A or C for 24 hours. C) Metabolic activity of A. fumigatus CEA10 biofilms (measured using resazurin) treated with increasing concentrations B or C for 24 hours. D) Propidium Iodide (PI) staining of A. fumigatus CEA10 hyphal cells treated with 16 μg/mL B for 24 hours. Images were acquired using the same exposure and magnification for all samples. Representative images from two independent experiments. For all graphs, data represent mean and SEM of three biological replicates and are normalized to untreated controls. IC₅₀ curves and values were calculated using GraphPad Prism 9.

FIG. 3. KAPs are efficacious in a rat model of C. albicans infection of a vascular catheter. A) Colony forming units (CFU) of C. albicans from central venous catheters placed in rats and treated with vehicle or A (8 μg/mL). Fungal burden data are shown as the mean and SEM of log-transformed CFU values; n=3 rats per group. **p<0.0001 by unpaired t-test of log-transformed values. B) Scanning electron micrographs of catheters treated with vehicle or 8 μg/mL A. Scale bar=10 μm.

FIG. 4. KAP A reduces C. auris colonization of skin in an ex vivo porcine model. Colony forming units CFUs of C. auris on porcine skin treated with vehicle or A (8 μg/mL or 16 μg/mL) for 24 hours. Data represent mean and SEM of log-transformed values; n=12 per group. **p<0.0001 by unpaired t-test of log-transformed values.

FIG. 5. Exemplary synthesis.

FIG. 6. Red blood cell and mammalian cell culture toxicity of KAP compounds. A. Hemolysis of commercial red blood cells (RBC) treated for 2 h with increasing concentrations of KAPs normalized to max lysis by triton-x. Mean and SD of technical triplicates. Representative data from three independent experiments performed on different days are shown. Toxicity against HepG2 cells using LDH release (B) and XTT (C) assays. Cells were treated with the indicated concentration series of A for 24 h. Mean and SD of technical triplicates. Data are representative of two independent experiments performed on different days.

FIG. 7. Synthesis of keto alkyl pyridiniums. A. PCl₅, 1.5 eq 150° C., 2 hours; B. 180° C., reflux, 0.5 hours. C. 2 hours, 100° C., acetonitrile.

FIG. 8. Keto-alkyl-pyridinium compounds are active against the yeast C. albicans in vitro and in two models of fungal disease. A) Select KAP compound structures and MIC values against the yeast C. albicans and the mold A. fumigatus. NT=Not tested. B) Fungal burden of catheters placed in a rat, seeded with C. albicans biofilm for 24 hours then treated with vehicle or 8 μg/mL Compound 3d for an additional 24 hours. n=3 mice per group. **p<0.0001 by unpaired t-test of log-transformed data. C) Fungal burden of porcine skin colonized with C. auris treated with 8 or 16 μg/mL Compound 3d for an additional 24 hours. n=12 samples per group. **p<0.0001 by unpaired t-test of log transformed data.

FIG. 9. Hemolysis of red blood cells (RBC) treated with escalating doses of KAP for 2 hours. Data are normalized to max lysis of RBC by Triton-X. LD₅₀ values are μg/mL.

### DETAILED DESCRIPTION

New antifungal therapies are needed for both systemic, invasive infections in addition to superficial infections of mucosal and skin surfaces as well as for biofilms associated with medical devices. The resistance of biofilm and biofilm-like growth phases of fungi contributes to the poor efficacy of systemic therapies to non-systemic infections. The identification and characterization of a keto-alkyl-pyridinium scaffold with broad spectrum activity (e.g., 2-16 μg/mL) against medically important yeasts and molds, including clinical isolates resistant to azoles and/or echinocandins, is disclosed herein. Furthermore, these keto-alkyl-pyridinium agents retain substantial activity against biofilm phase yeast and have direct activity against hyphal A. fumigatus. It was found that the keto-alkyl-pyridinium molecules reduce Candida albicans fungal burden in a rat model of vascular catheter infection and reduce Candida auris colonization in a porcine ex vivo model. These data suggest that molecules of this class may be useful in at least non-systemic applications.

### Definitions

The chemical genera provided herein are intended to be understood as describing “chemically feasible” structures, by which is meant that the structure depicted by any combination or subcombination of optional substituents meant to be recited by the claim is physically capable of existence with at least some stability as can be determined by the laws of structural chemistry and by experimentation. Structures that are not chemically feasible are not within a claimed set of compounds.

The term “alkyl” as used herein refers to substituted or unsubstituted straight chain, branched, or cyclic, saturated hydrocarbon group. The group can have from 1 to 10 carbon atoms, 1 to 6 carbon atoms, 1 to 4 carbon atoms, 2 to 6 carbon atoms, or 2 to 4 carbon atoms. Examples of straight chain alkyl groups include methyl (i.e., CH3), ethyl, n-propyl, n-butyl, n-pentyl, n-hexyl, n-heptyl, n-octyl groups. Examples of branched alkyl include isopropyl, iso-butyl, sec-butyl, t-butyl, neopentyl, and isopentyl. An alkyl can be optionally substituted. The term “alkoxy” refers to an alkyl linked to a divalent oxygen. The term “alkylthio” refers to an alkyl linked to a divalent sulfur.

The term “aryl” as used herein refers to a aromatic hydrocarbon groups and heterocyclic aromatic groups. The group can have from 6 to about 10 atoms, for example 5 atoms, 6 atoms, or 10 atoms. Examples include phenyl, naphthyl, and heteroaryl. An aryl can be optionally substituted. The term “heteroaryl” or “hetaryl” as used herein refers to an aromatic heterocyclic group. The group can have from a ring size of 5 to 10 atoms, 5 to 9 atoms, or 5 to 6 atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzirnidazolyl, azabenzirnidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, tetrahydroisoquinolinyl, quinoxalinyl, and quinazolinyl groups. The terms “heteroaryl” and “heteroaryl groups” include fused ring compounds such as wherein at least one ring, but not necessarily all rings, are aromatic, including tetrahydroquinolinyl, tetrahydroisoquinolinyl, indolyl and 2,3-dihydro indolyl. A heteroaryl can be optionally substituted. The term “aryloxy” refers to an aryl linked to a divalent oxygen. The term “arylthio” refers to an aryl linked to a divalent sulfur. The term “arylmethyl” refers to an aryl linked to a divalent methylene. The term “arylmethoxy” refers to an aryl linked to a divalent methylene further linked to a divalent oxygen. The term “phenylene” refers to a divalent benzene moiety, which includes a 1,4-phenylene, 1,3-phenylene, and 1,2-phenylene.

In general, “substituted” and “substituent” refers to an organic group as defined herein in which one or more bonds to a hydrogen atom contained therein are replaced by one or more bonds to a non-hydrogen atom such as, but not limited to, a halogen (i.e., “halo” selected from F, Cl, Br, and I); an oxygen atom in groups such as hydroxyl groups, alkoxy groups, aryloxy groups, aralkyloxy groups, oxo (carbonyl) groups, carboxyl groups including carboxylic acids, carboxylates, and carboyxlate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR′, OC(O)N(R′)2, CN, CF3, OCF3, R′, O, S, C(O), S(O), methylenedioxy, ethylenedioxy, N(R′)2SR′, SOR′, SO2R′, SO1N(R′)2, SO3R′, C(O)R′, C(O)C(O)R′, C(O)CH2C(O)R′, C(S)R′, C(O)OR′, OC(O)R′, C(O)N(R′)2, OC(O)N(R′)2, C(S)N(R′)2, (CH2)O-2NHC(O)R′, (CH2)0-2N(R′)N(R′)2, N(R′)N(R′)C(O)R′, N(R′)N(R′)C(O)OR′, N(R′)N(R′)CON(R′)2, N(R′)SO2R′, N(R′)SO2N(R′)2, N(R′)C(O)OR′, N(R′)C(O)R′, N(R′)C(S)R′, N(R′)C(O)N(R′)2, N(R′)C(S)N(R′)2, N(COR′)COR′, N(OR′)R′, C(═NH)N(R′)2, C(O)N(OR′)R′, or C(═NOR′)R′ wherein R′ can be hydrogen or a carbon-based moiety, and wherein the carbon-based moiety can itself be further substituted. For example, R′ group can be hydrogen, C1-C6 alkyl, or phenyl.

A “salt” as is well known in the art includes an organic compound such as a pyridinium or ammonium, or a pyridine or amine in ionic form, in combination with a counterion. For example, pyridinium ions exist in salt form with a counterion anion such as a halide, a carboxylate or sulfonate. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein. The compounds of the present disclosure may take the form of salts. The term “salts” embraces addition salts of free acids or free bases which are compounds. Salts can be “pharmaceutically-acceptable salts.” The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present disclosure, such as for example utility in process of synthesis, purification or formulation of compounds.

Suitable pharmaceutically-acceptable acid addition salts may be prepared from an inorganic or an organic conjugate base. Examples of inorganic acids include chloride, bromide, iodide, nitrate, nitrite, carbonate, sulfonate, or phosphate. Example organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates.

The term “pharmaceutically acceptable salts” refers to nontoxic inorganic or organic acid and/or base addition salts, see, for example, Lit et al., Salt Selection for Basic Drugs (1986), Int J. Pharm., 33, 201-217, incorporated by reference herein.

The term “killing” in the present application can distinguish between compounds that kill fungal cells versus those that stop fungal growth. Compounds that kill fungul cells are important because patients who develop fungal infections can be immune deficient and do not have adequate immune activity to help resolve infections.

As used herein, by “administering” is meant a method of giving a dosage of a composition described herein to a subject. The compositions utilized in the methods described herein can be administered by any suitable route, including, for example, by inhalation, nebulization, aerosolization, intranasally, intratracheally, intrabronchially, orally, parenterally (e.g., intravenously, subcutaneously, or intramuscularly), orally, nasally, rectally, topically, or buccally. In some embodiments, a composition described herein is administered in aerosolized particles intratracheally and/or intrabronchially using an atomizer sprayer (e.g., with a MADgic® laryngo-tracheal mucosal atomization device). The compositions utilized in the methods described herein can be administered locally or systemically. The method of administration can vary depending on various factors (e.g., the components of the composition being administered and the severity of the condition being treated).

The terms “subject” and “patient” are used interchangeably herein to refer to any mammal (e.g., a human, a primate, a cat, a dog, a ferret, a cow, a horse, a pig, a goat, a rat, or a mouse). For example, the subject is a human.

By “therapeutically effective amount” is meant the amount of a composition administered to improve, inhibit, or ameliorate a condition of a subject, or a symptom of a disorder or disease, in a clinically relevant manner. Any improvement in the subject is considered sufficient to achieve treatment. In one embodiment, an amount sufficient to treat is an amount that reduces, inhibits, or prevents a fungal infection or is an amount that reduces the severity of, or the length of time during which a subject suffers from, one or more symptoms of the infection (e.g., by at least about 10%, about 20%, or about 30%, or by at least about 50%, about 60%, or about 70%, or by at least about 80%, about 90%, about 95%, about 99%, or more, relative to a control subject that is not treated with a composition described herein). An effective amount of the pharmaceutical composition used to practice the methods described herein varies depending upon the manner of administration and the age, body weight, and general health of the subject being treated. A physician or researcher can decide the appropriate amount and dosage regimen.

In particular, a “therapeutically effective amount” refers to an amount effective, at dosages and for periods of time necessary, to achieve the desired therapeutic result. A therapeutically effective amount is also one in which any toxic or detrimental effects are outweighed by the therapeutically beneficial effects.

As used herein, an “effective amount” refers to an amount that alleviates, in whole or in part, symptoms associated with the infection, or halts or slows further progression or worsening of those symptoms, or prevents or provides prophylaxis for the infection, e.g., an amount that is effective to prevent, inhibit or treat the infection in an individual and/or alleviate one or more symptoms of the infection.

For example, “treating” or “treatment” within the meaning herein refers to at least inhibiting and optionally eliminating or eradicating the infection, “inhibiting” means inhibition of the infection, and “preventing” refers to prevention of an infection

### General Processes for Making Compounds of the Disclosure

The present disclosure also includes processes for the preparation of compounds of the disclosure. A general approach is provided to obtain various compounds of the present disclosure. In the reactions described, where justified based on the inclusion of reactive functional groups, it can be necessary to protect such groups, for example hydroxy, amino, imino, thio or carboxy groups, and where these are desired in the final product, to avoid their unwanted participation in the reactions. Conventional protecting groups can be used in accordance with standard practice, for example, see T. W. Greene and P. G. M. Wuts in “Protective Groups in Organic Chemistry”, John Wiley and Sons, 1991.

The compounds of the disclosure can be produced by organic synthesis methods known to one of ordinary skill in the art with reference to the following reaction schemes and examples. Example general synthetic approaches are provided below.

### General Synthetic Approach Example 1

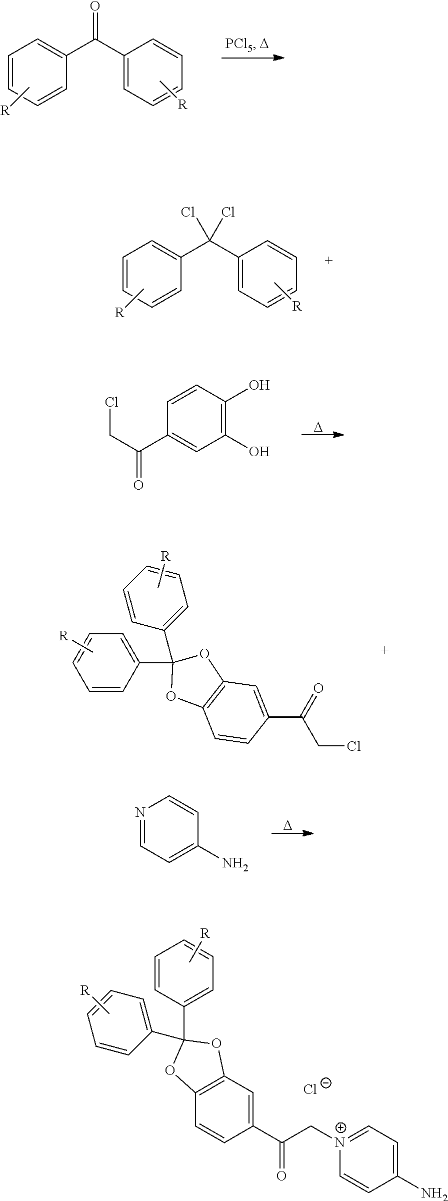

### General Synthetic Approach Example 2

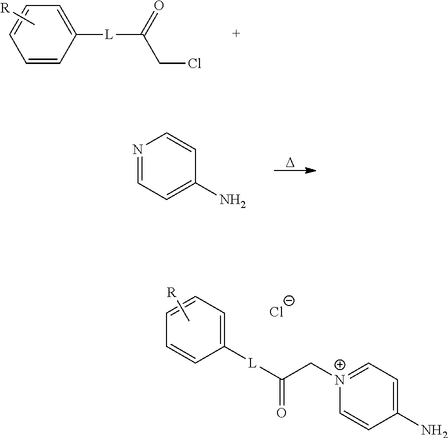

The first example approach provides access to ketal-containing products while the second example approach provides more a streamlined approach for compounds where the desired phenyl substitution pattern is readily available as a starting materials. While the general approaches illustrate use of chlorides, ketones, and phenyl groups, a person skilled in the art would understand that alternative functional groups can also be employed. The ketal compounds of the present disclosure can be prepared a suitable starting ketone compound, such as benzophenone or a derivative thereof, treated with a halogenation agent and/or heat. The resulting geminal dihalo compound is mixed with an alpha-substituted acetophenone, or derivative thereof, that contains two adjacent hydroxyl groups. The mixture is heated to provide a ketal reagent primed for reaction with a pyridine. Further combination with 4-aminopyridine and heat provides the target pyridinium salt. Where alpha-haloketone starting materials are readily available, they may be readily combined with pyridine derivatives to provide the desired pyridinium salt. Further detailed descriptions of synthesis of compounds are given in examples, infra.

### Synthesis of Keto-Alkyl-Pyridinium (KAP) Compounds

### Materials and Methods

### Reagents and Instrumentation.

Reagents were purchased as at least reagent grade from Aldrich, Acros or Alfa Aesar unless otherwise specified and used without further purification. Solvents were purchased from Fischer Scientific (Pittsburgh, PA) and were either used as purchased or redistilled with an appropriate drying agent. Compounds used for structure-activity studies were synthesized according to methods described below, and all the compounds were identified to be least 95% pure using HPLC. Analytical TLC was performed using precoated Silica G TLC Plates, w/UV254, aluminum backed, purchased from Sorbtech (Norcross, GA) and visualized using UV light. Flash chromatography was carried out using with a Biotage Isolera One (Charlotte, NC) system using the specified solvent. Microwave reactions were performed using Biotage Initiator+ (Charlotte, NC). Purity analysis were performed on an Agilent 1100 HPLC utilizing a C-18 column (Waters Nova-Pak; 3.9×100 mm) with the following method: Solvent A=H2O (0.1% TFA), Solvent B=Acetonitrile; 0 to 20 min, (10 to 90% B), 20 to 25 min (90 to 10% B); detection was set at two wavelengths (245 and 280 nm). Purity of all final compounds was above 95%. All final compounds were analyzed by high resolution MS (HRMS) using a Bruker Maxis Plus Quadrupole Time-of-Flight (QTOF or QqTOF). 1H and 13C NMR were recorded on either a BrukerAvance III 500 outfitted with a 5 mm BBFO Z-gradient probe or a Avance III 300 instrument is equipped with a 5 mm BBFO probe. The chemical shifts are expressed in parts per million (ppm) using suitable deuterated NMR solvents.

The presently described keto-alkyl-pyridinium compounds represent a structurally novel extension of pyridinium class antiseptics that have broad spectrum antifungal activity. A set of compounds exhibiting the keto-alkyl-pyridinium structural aspect is exemplified in structures 3a-d (Scheme 1). Previous pyridinium agents have alkyl substituents whereas the present compounds have aryl ketone substituents. In addition, various presently described KAP compounds contain a dioxolane ring system that represents a further class of antifungal KAP. In addition to the structurally unique features, we have shown that these molecules are active against a broad range of medically important fungi. Finally, we have shown these molecules have activity against C. albicans biofilms in an in vivo rat catheter model and are more active than chlorhexidine against C. auris skin infection using an ex vivo model of this process.

Compounds 3a-d were synthesized at Northern Illinois University. The synthesis is outlined in Scheme 1. These are easily generated in a three-step synthesis and were purified to >95% purity.

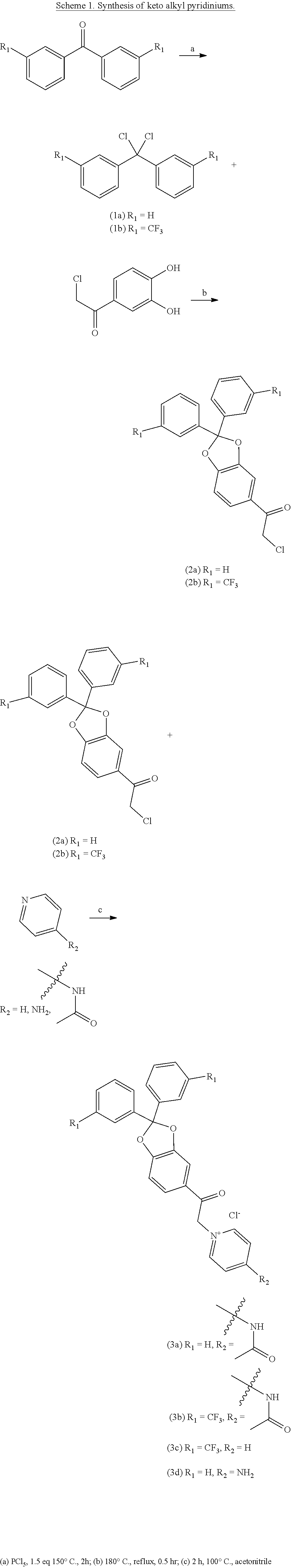

Dichlorodiphenylmethane (1). To a round flask was weighed benzophenone (1 eq) and PCl₅ (1.5 eq). The reaction was refluxed for 2 h at 150° C. After 2 hours, the reaction was cooled, 40 mL of DCM was added to the resultant solution followed by transferring to 250 mL Erlenmeyer flask. The resultant solution was cooled to 0° C. and 40 mL of distilled water was added. The resultant solution was transferred to a separatory funnel, the DCM layer was separated, washed twice with 40 ml distilled water, and dried with anhydrous sodium sulfate. The DCM solvent was evaporated in rotary evaporator to give corresponding dichloride. The crude product was immediately used for the next step without purification.

2-Chloro-1-(2,2-diphenylbenzo[d][1,3]dioxol-5-yl)ethenone (2). To a round flask was weighed dichlorodiphenylmethane (1 g, 4.2 mmol) and 2-chloro-1-(3,4-dihydroxyphenyl) ethanone (0.79 g, 4.2 mmol) under nitrogen. The reaction was refluxed at 180° C. for 30 mins under nitrogen. After 30 mins. The crude product was transferred to silica gel chromatography with linear gradient of 2-10% (EtOAc/Hexane). The fractions corresponding to the desired product were collected and solvent evaporated using rotatory evaporator to give yellow viscous liquid as the desired product which turned to a solid upon dryness. Yield; 0.7 g, 47%, 1H NMR (500 MHz, CDCl₃) δ 7.69 (m, 4H), 7.61-7.57 (m, 2H), 7.44 (m, 6H), 6.98 (d, J=7.9 Hz, 1H), 4.63 (s, 2H). 13C NMR (75 MHz, CDCl₃), 189.26, 152.05, 148.05, 139.46, 129.56, 129.01, 128.41, 125.14, 118.58, 108.52, 108.38, 45.84.

4-Acetamido-1-(2-(2,2-diphenylbenzo[d][1,3]dioxol-5-yl)-2-oxoethyl)pyridin-1-ium chloride (3). To a round flask containing 2-chloro-1-(2,2-diphenylbenzo[d][1,3]dioxol-5-yl)ethenone (0.34 0.97 mmol) dissolved in acetonitrile (10 mL) was weighed the 4-aminopyridine (0.09 g, 0.97 mmol). The reaction was refluxed for 2 hrs at 100° C. in which precipitate was formed. After 2 hrs, the solvent was evaporated using rotatory evaporator and the gummy solid precipitated with 10% Hexane in DCM, filtered and dried to give a powdered solid product in form of the chloride salt. HPLC purity; 98%. Yield; 0.38 g, 88%, 1H NMR (300 MHz, DMSO) δ 12.12 (s, 1H), 8.65 (d, J=6.9 Hz, 2H), 8.22 (d, J=6.9 Hz, 2H), 7.74 (d, J=8.3 Hz, 2H), 7.68 (s, 1H), 7.57-7.55 (m, 4H), 7.48-7.32 (m 6H), 7.32 (d, J=8.2 Hz, 1H), 6.21 (s, 2H), 2.27 (s, 3H). 13C NMR (75 MHz, DMSO), 189.88, 171.71, 152.87, 151.87, 147.59, 146.99, 139.30, 130.21, 129.18, 129.00, 126.25, 125.70, 118.36, 114.77, 109.56, 108.65, 64.75, 25.06, HRMS for [M+H]+ calculated; 451.1652, found; 451.1658.

### Exemplary Compounds

TABLE 1
Example Compounds

| | |
|---|---|
| 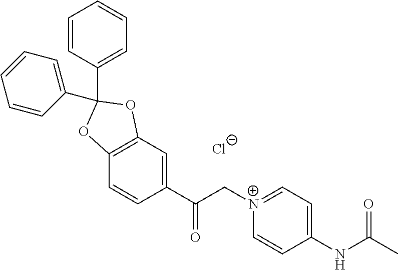 [SMILES: CC(=O)Nc1cc[n+](CC(=O)c2ccc3c(c2)OC(c2ccccc2)(c2ccccc2)O3)cc1 ; [Cl-]] | Compound 3a |
| 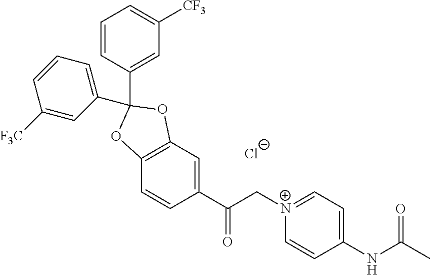 [SMILES: CC(=O)Nc1cc[n+](CC(=O)c2ccc3c(c2)OC(c2cccc(C(F)(F)F)c2)(c2cccc(C(F)(F)F)c2)O3)cc1 ; [Cl-]] | Compound 3b |
| 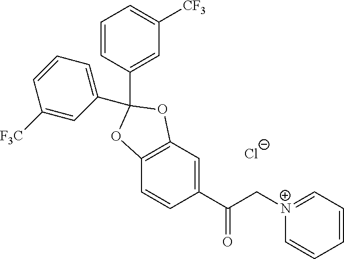 [SMILES: O=C(C[n+]1ccccc1)c1ccc2c(c1)OC(c1cccc(C(F)(F)F)c1)(c1cccc(C(F)(F)F)c1)O2 ; [Cl-]] | Compound 3c |
| 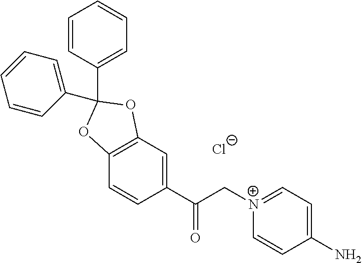 [SMILES: Nc1cc[n+](CC(=O)c2ccc3c(c2)OC(c2ccccc2)(c2ccccc2)O3)cc1 ; [Cl-]] | Compound 3d/Compound A |
| 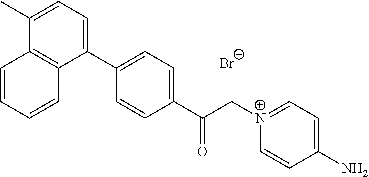 [SMILES: Cc1ccc(-c2ccc(C(=O)C[n+]3ccc(N)cc3)cc2)c2ccccc12 ; [Br-]] | Compound 4a |
| 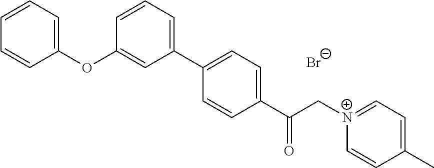 [SMILES: Cc1cc[n+](CC(=O)c2ccc(-c3cccc(Oc4ccccc4)c3)cc2)cc1 ; [Br-]] | Compound 4b |
| 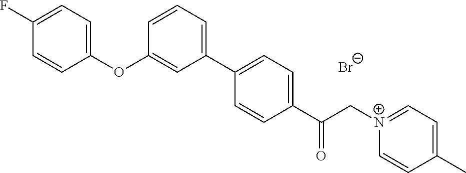 [SMILES: Cc1cc[n+](CC(=O)c2ccc(-c3cccc(Oc4ccc(F)cc4)c3)cc2)cc1 ; [Br-]] | Compound 4c |
| 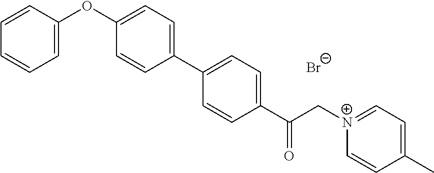 [SMILES: Cc1cc[n+](CC(=O)c2ccc(-c3ccc(Oc4ccccc4)cc3)cc2)cc1 ; [Br-]] | Compound B |
| 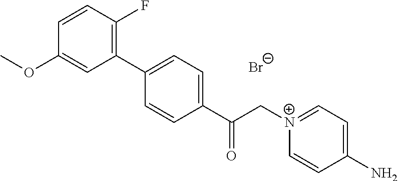 [SMILES: COc1ccc(F)c(-c2ccc(C(=O)C[n+]3ccc(N)cc3)cc2)c1 ; [Br-]] | Compound C (control) |
| 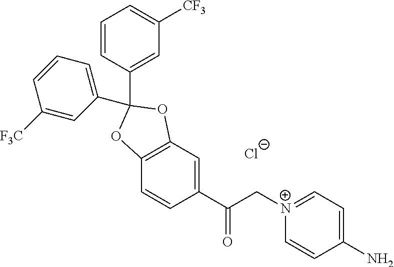 [SMILES: Nc1cc[n+](CC(=O)c2ccc3c(c2)OC(c2cccc(C(F)(F)F)c2)(c2cccc(C(F)(F)F)c2)O3)cc1 ; [Cl-]] | KAP-1 |
| 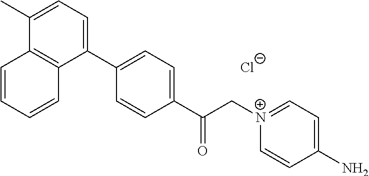 [SMILES: Cc1ccc(-c2ccc(C(=O)C[n+]3ccc(N)cc3)cc2)c2ccccc12 ; [Cl-]] | KAP-2 |
| 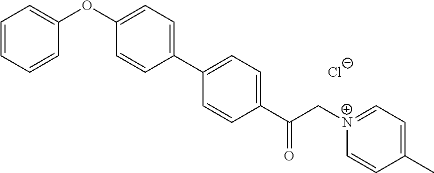 [SMILES: Cc1cc[n+](CC(=O)c2ccc(-c3ccc(Oc4ccccc4)cc3)cc2)cc1 ; [Cl-]] | KAP-3 |
| 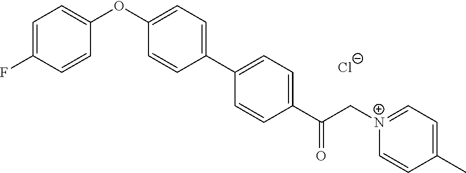 [SMILES: Cc1cc[n+](CC(=O)c2ccc(-c3ccc(Oc4ccc(F)cc4)cc3)cc2)cc1 ; [Cl-]] | KAP-4 |
| 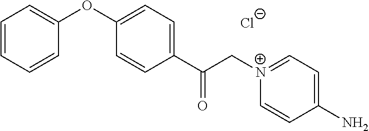 [SMILES: Nc1cc[n+](CC(=O)c2ccc(Oc3ccccc3)cc2)cc1 ; [Cl-]] | KAP-5 |

### Compositions Comprising KAP Compounds

A composition having one or more of the disclosed compounds can be formulated as a pharmaceutical composition and administered to a mammalian host, such as a human patient in a variety of forms adapted to the chosen route of administration. In one embodiment, the composition is locally administered. In one embodiment, the composition is administered prophylactically. The composition may include a delivery vehicle such as a liposome, dendrimer, nanoparticle or microparticle.

In one embodiment, the composition may be administered by injection. Solutions may be prepared in water, optionally mixed with a nontoxic surfactant. Dispersions may also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection may include sterile aqueous solutions or dispersions or sterile powders comprising the active ingredient which are adapted for the extemporaneous preparation of sterile solutions or dispersions. In all cases, the ultimate dosage form should be sterile, fluid and stable under the conditions of manufacture and storage. The liquid carrier or vehicle may be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. In many cases, isotonic agents, for example, sugars, buffers or sodium chloride may be included.

Sterile solutions may be prepared by incorporating the ketoalkylpyridinium compounds, e.g., with aryl substitutions, in the desired amount in the appropriate solvent with various other ingredients, as required. In the case of sterile powders for the preparation of sterile injectable solutions, the methods of preparation include vacuum drying and the freeze drying techniques, which yield a powder of the active ingredient plus any additional desired ingredient present in the previously sterile-filtered solutions.

Useful solid carriers may include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants can be added to optimize the properties for a given use. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compound(s) in the composition can be determined by comparing their in vitro activity and in vivo activity in animal models thereof. Methods for the extrapolation of effective dosages in mice, and other animals, to humans are known to the art; for example, see U.S. Pat. No. 4,938,949.

The concentration of the therapeutically or prophylactically effective compound(s) in a composition, may be from about 0.1-25 wt-%, e.g., from about 0.5-10 wt-% or may be about 0.1-5 wt-%, e.g., about 0.5-2.5 wt-%.

The amount of the therapeutically or prophylactically effective compound for use alone or with other agents may vary with the composition of the polymer, route of administration, the nature of the condition being treated and the age and condition of the patient and will be ultimately at the discretion of the attendant physician or clinician.

### Pharmaceutical Compositions

The disclosure provides a composition comprising, consisting essentially of, or consisting of the ketoalkylpyridinium compounds, e.g., with aryl substitutions, optionally in a delivery vehicle, e.g., one or more particle(s), and/or optionally a pharmaceutically acceptable (e.g., physiologically acceptable) carrier. In one embodiment, when the composition consists essentially of one or more ketoalkylpyridinium compounds, e.g., with aryl substitutions, and a pharmaceutically acceptable carrier, optionally additional components can be included that do not materially affect the composition (e.g., adjuvants, buffers, stabilizers, anti-inflammatory agents, solubilizers, preservatives, etc.). In one embodiment, when the composition consists of the one or more ketoalkylpyridinium compounds, e.g., with aryl substitutions, and optionally a pharmaceutically acceptable carrier, the composition does not comprise any additional components. Any suitable carrier can be used, and such carriers are well known in the art. The choice of carrier will be determined, in part, by the particular site to which the composition may be administered and the particular method used to administer the composition. The compositions can be generated in accordance with conventional techniques described in, e.g., Remington: The Science and Practice of Pharmacy, 21st Edition, Lippincott Williams & Wilkins, Philadelphia, PA (2001).

Suitable formulations for the composition include aqueous and non-aqueous solutions, isotonic sterile solutions, which can contain anti-oxidants, buffers, or bacteriostats, or combinations thereof, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. In one embodiment, the carrier is a buffered saline solution. The composition may comprise a pharmaceutically acceptable liquid carrier, such as, for example, those described above, and a stabilizing agent selected from the group consisting of polysorbate 80, L-arginine, polyvinylpyrrolidone, trehalose, and combinations thereof.

In addition, one of ordinary skill in the art will appreciate that the composition comprising one or more ketoalkylpyridinium compounds, e.g., with aryl substitutions, can be present in a composition with other therapeutic or biologically-active agents. For example, factors that control inflammation, such as ibuprofen or steroids, can be part of the composition to reduce swelling and inflammation associated with in vivo administration. Immune system stimulators or adjuvants, e.g., interleukins, lipopolysaccharide, and double-stranded RNA. Antibiotics, i.e., other microbicides and fungicides, can be present to treat existing infection and/or reduce the risk of future infection.

Injectable depot forms comprising biodegradable polymers such as polylactide-polyglycolide may be employed. Depending on the ratio of components in the polymer, and the nature of the particular polymer employed, the rate of drug release can be controlled. Examples of other biodegradable polymers include poly(orthoesters) and poly(anhydrides). Depot injectable formulations are also envisioned which are compatible with body tissue.

In certain embodiments, a formulation comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho) esters, poly(butic acid), poly(valeric acid), poly(lactide-co-caprolactone), polysaccharides, proteins, polyhyaluronic acids, polycyanoacrylates, and blends, mixtures, or copolymers thereof.

The composition may be administered in or on a device that allows controlled or sustained release, such as a sponge, biocompatible meshwork, or mechanical reservoir.

The dose of the ketoalkylpyridinium compounds, e.g., with aryl substitutions, in the composition administered to the mammal will depend on a number of factors, including the size (mass) of the mammal, the extent of any side-effects, the particular route of administration, and the like. In one embodiment, the method comprises administering a “therapeutically effective amount” of the composition. A “therapeutically effective amount” refers to an amount effective, at dosages and for periods of time necessary, to achieve a desired therapeutic result. The therapeutically effective amount may vary according to factors such as the extent of the disease or disorder, age, sex, and weight of the individual.

### Routes of Administration, Dosages and Dosage Forms

Both local administration and systemic administration are contemplated. In one embodiment, a composition may be delivered to the skin or eye. One or more suitable unit dosage forms comprising one or more ketoalkylpyridinium compounds, e.g., with aryl substitutions, can be administered by a variety of routes including local. The formulations may, where appropriate, be conveniently presented in discrete unit dosage forms and may be prepared by any of the methods well known to pharmacy. Such methods may include the step of bringing into association the subunit components, e.g., subunits of a polymer or co-polymer, or the polymer or co-polymer, and the drug and optionally liquid carriers, solid matrices, semi-solid carriers, finely divided solid carriers or combinations thereof, and then, if necessary, introducing or shaping the product into the desired delivery system.

The biocompatible delivery vehicle such as one or more particle(s) may conveniently be provided in the form of formulations suitable for administration. A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. By “pharmaceutically acceptable” it is meant a carrier, diluent, excipient, and/or salt that is compatible with the other ingredients of the formulation, and not deleterious to the recipient thereof.

The biocompatible delivery vehicle may be formulated in solution at neutral pH, for example, about pH 6.5 to about pH 8.5, or from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, or from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is useful for buffers containing sodium ions. If desired, solutions of the above compositions can also be prepared to enhance shelf life and stability. Therapeutically useful compositions can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

In one embodiment, a biocompatible delivery vehicle such as one or more particle(s) may be formulated for administration, e.g., by injection or via a catheter, and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulary agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint.

Thus, the local delivery of the biocompatible delivery vehicle, such as one or more particle(s), can be by a variety of techniques which administer the one or more particle(s) at or near the site of infection, e.g., using a catheter or needle. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

The formulations and compositions described herein may also contain other ingredients such as other antimicrobial agents or preservatives.

By way of illustration, liposomes and other lipid-containing delivery complexes may be employed as a delivery vehicle.

The pharmaceutical formulations can also take the form of an aqueous or anhydrous solution, e.g., a lyophilized formulation, or dispersion, or alternatively the form of an emulsion or suspension.

In one embodiment, the formulation is a gel, cream, foam, lotion, oil, ointment, paste, suspension, or aerosol spray comprising, for example, 5-10 wt % of a ketoalkylpyridinium compound, e.g., with an aryl substitution.

### Exemplary Formulations for Topical Administration and Dosages

The ketoalkylpyridinium compounds, e.g., with aryl substitutions, can be formulated as pharmaceutical compositions and topically administered to a mammalian host, such as a human patient. In one embodiment, a sustained release formulation comprising the ketoalkylpyridinium compounds, e.g., with aryl substitutions, may be topically administered. Solutions of the ketoalkylpyridinium compounds, e.g., with aryl substitutions, or salts thereof, can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms for topical application may include sterile aqueous solutions or dispersions or sterile powders comprising the active ingredient which are adapted for the extemporaneous preparation of sterile solutions or dispersions, optionally encapsulated in nanoparticles, e.g., liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the particle size in the case of dispersions or by the use of surfactants. In many cases, it may be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile solutions are prepared by incorporating the active agent in the desired amount in the appropriate solvent with various of the other ingredients enumerated above, as required, followed by, for example, filter sterilization. In the case of sterile powders for the preparation of sterile solutions, the methods of preparation include vacuum drying and the freeze drying techniques, which yield a powder of the active ingredient plus any additional desired ingredient present in the previously sterile-filtered solutions.

Useful solid carriers may include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants can be added to optimize the properties for a given use. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

The amount of the ketoalkylpyridinium compounds, e.g., with aryl substitutions, for use alone or with other agents will vary with the nature of the condition being treated and the age and condition of the patient and will be ultimately at the discretion of the attendant physician or clinician.

### Penetration Enhancers

The compositions described herein can optionally include one or more penetration enhancers. The penetration enhancer can be, for example, a pharmaceutically acceptable inorganic or an organic base. Examples of inorganic bases include inorganic hydroxides, inorganic oxides, inorganic salts of weak acids, and combinations thereof. Suitable organic bases can include one or more nitrogenous bases.

The body surface can be exposed to a composition described herein that includes a penetration enhancer, such as a base or basic solution or other agent described below, for a sufficient period of time so as to provide a high pH at the skin surface (e.g., a pH of about 7.5 to about 12), thus creating channels in the skin or mucosa for the drug to pass through. The drug flux can be proportional to the strength of the solution and the duration of exposure. The composition can also include at least one irritation-mitigating additive.

Examples of classes of enhancers include, but are not limited to, fatty acids, saturated and/or unsaturated; fatty alcohols; bile acids; nonionic surfactants, including esters of fatty acids, fatty (long-chain alkyl or alkenyl) esters of monohydric alcohols, diols, and polyols, diols and polyols that are both esterified with a fatty acid and/or substituted with a polyoxyalkylene, polyoxyalkylene fatty acid esters, polyoxyalkylene fatty ethers, polyoxyalkylene fatty ethers, and polyglyceryl fatty acid esters; amines; amides; N-alkyl-azacycloalkanones; hydrocarbon solvents; terpenes; lower alkyl esters; cyclodextrin enhancers; nitrogen-containing heterocycles; sulfoxides; urea and its derivatives, or combinations thereof.

Specific examples of suitable enhancers include ethers such as diethylene glycol monoethyl ether (available commercially as Transcutol®, Gattefosse S A) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80) and lecithin; alcohols such as ethanol, propanol, octanol, benzyl alcohol, and the like; fatty acids such as lauric acid, oleic acid and valeric acid; fatty acid esters such as isopropyl myristate, isopropyl palmitate, methylpropionate, and ethyl oleate; polyols and esters thereof such as polyethylene glycol, and polyethylene glycol monolaurate; amides and other nitrogenous compounds such as urea, dimethylacetamide, dimethylformamide, 2-pyrrolidone, 1-methyl-2-pyrrolidone, ethanolamine, diethanolamine and triethanolamine; terpenes; alkanones; and organic acids, particularly citric acid and succinic acid. Azone® and sulfoxides such as dimethylsulfoxide and decylmethylsulfoxide may also be used, but are less preferred. Combinations of one or more of the preceding agents can be added to a composition described herein. Percutaneous Penetration Enhancers, eds. Smith et al. (CRC Press, 1995) provides an overview of the field and further information concerning suitable secondary enhancers for use in conjunction with the compositions described herein. Various suitable lipophilic co-enhancers are also described in U.S. Pat. No. 6,835,392 (Hsu et al.) and U.S. Pat. No. 7,205,003 (Maibach et al.). Additional penetration enhancers that can be included are described by Ehdaie (J. Pharm. Biomed. Sci., 1 (8), 161-166, 2011).

The composition may also contain irritation-mitigating additives to minimize or eliminate the possibility of skin irritation or damage to sensitive skin resulting from the drug, the base enhancer, or other components of the formulation. Suitable irritation-mitigating additives include, for example: α-tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; chloroquine, or combinations thereof. The irritant-mitigating additive, if present, may be incorporated into the formulation at a concentration effective to mitigate irritation or skin damage, typically representing not more than about 20 wt. %, more typically not more than about 5 wt. %, of the formulation, and often about 0.1 to about 3 wt. % of the formulation.

In some embodiments, dimethyl sulfoxide (DMSO) can be included in the composition as a penetration enhancer. DMSO is a rapidly absorbed skin penetrant, with the ability to carry other substances through membranes such as skin, while it acts as an anti-inflammatory agent. Active agents dissolved in DMSO retain their therapeutic activity and their specific properties over a prolonged period of time; it can maintain and multiply the activity of the active it carries. Allergic reactions can be diminished by the inclusion of DMSO. Any detectable order of DMSO can be diminished, for example, by using aloe or a mint fragrance.

Pharmaceutical formulations will typically include the therapeutic composition, a pharmaceutically acceptable carrier, and optionally one or more additional ingredients that, for example, aid the formation of the desired delivery vehicle of the active. For topical administration, it will generally be desirable to administer the active agent to the skin as a composition or formulation, for example, in combination with a dermatologically acceptable carrier, where the composition or formulation may be a semi-solid, oil, or a liquid. Liquid compositions can be applied from absorbent pads, used to impregnate bandages and other dressings, or sprayed onto the affected area using a pump-type or aerosol sprayer.

A topical composition typically includes an active and a pharmaceutically acceptable carrier for topical administration. The administration can be the topical application of a gel, a jelly, a cream, a lotion, a wax, an ointment, a solution, a paste, an aerosol, a patch, and/or a combination thereof. Suitable pharmaceutically acceptable carriers include, but are not limited to, creams such as Cetaphil Moisturising Cream (Galderma Laboratories, L.P.), QV Cream (Lision Hong), Sorbolene, or the like. In some embodiments, the pharmaceutical acceptable carrier includes a lotion, such as Alpha Ken Moisturizing Lotion (Mentholatum), Derma Veen Moisturizing Lotion (DermaTech Laboratories), QV Skin Lotion (Lision Hong), Cetaphil Moisturizing Lotion (Galderma Laboratories, L.P.), or the like. In any embodiment, the pharmaceutically acceptable carrier can include one or more oils.

### Salts.

In cases where actives are sufficiently basic or acidic to form stable nontoxic acid or base salts, administration of the actives as salts may be appropriate. Examples of pharmaceutically acceptable salts are organic acid addition salts formed with acids which form a physiological acceptable anion, for example, tosylate, methanesulfonate, acetate, propionate, citrate, malonate, tartrate, succinate, benzoate, ascorbate, α-ketoglutarate, and α-glycerophosphate. Pharmaceutically acceptable salts can also be mineral acid salts such as hydrochlorides, hydrobromides, and the like. Suitable salts may also be formed as halides, nitrates, phosphates, sulfates, bicarbonates, carbonate salts, and the like.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

### Carriers.

Active agents can be combined with a pharmaceutically acceptable carrier or diluent to produce a pharmaceutical composition or formulation. In such pharmaceutical formulations, the active agents or therapeutic composition can be combined with a “carrier” that is physiologically compatible with the skin or mucosal tissue of a human or animal to which it is topically administered. Typically the carrier is substantially inactive, with the exception of its intrinsic surfactant properties which may aid in the production of a solution or suspension of the active ingredients. The compositions may include other physiologically active constituents that do not interfere with the efficacy of the active agents in the composition. In some embodiments, the carriers can be liquid or gel-based materials for use in liquid or gel formulations. The specific formulations depend, in part, upon the desired routes or modes of administration.

Suitable carrier materials include any carrier or vehicle commonly used as a base for solutions, dispersions, emulsions, gels, creams, ointment, lotions, pastes, or foams, for topical administration. Examples include emulsifying agents, inert carriers including hydrocarbon bases, emulsifying bases, non-toxic solvents or water-soluble bases.

Many suitable liquid or gel-based carriers are well-known in the art. The carrier should be able to dissolve or disperse an active at an effective level, optionally with the aid of non-toxic surfactants. Examples include water, physiological salt solutions, alcohols (e.g., methanol, ethanol, propanol, or butanol), glycerol, glycols (e.g., ethylene glycol, propylene glycol, or ethoxy diglycol), polyethylene glycol (e.g., MW 400 to 20,000), water-alcohol/glycol blends, and the like. Suitable carriers and diluents for certain embodiments include, for example, water, saline, isotonic saline solutions, for example, phosphate-buffered saline, aqueous dextrose, glycerol, ethoxy diglycol, dimethyl sulfoxide (DMSO), and the like, or combinations thereof.

Suitable carriers further include aqueous and oleaginous carriers such as, for example, white petrolatum, isopropyl myristate, lanolin or lanolin alcohols, mineral oil, fragrant or essential oil, nasturtium extract oil, sorbitan mono-oleate, cetostearyl alcohol (together or in various combinations), and detergents (e.g., polysorbates (Tweens) such as polysorbate 20, 40, 60, or 80; polyoxyl stearate; or sodium lauryl sulfate). One or more carrier materials can be mixed with water to form a lotion, gel, cream, semi-solid composition, or the like. Other suitable carriers include water-in-oil or oil-in-water emulsions and mixtures of emulsifiers and emollients with solvents such as sucrose stearate, sucrose cocoate, sucrose distearate, mineral oil, propylene glycol, 2-ethyl-1,3-hexanediol, polyoxypropylene-15-stearyl ether, water, or combinations thereof. For example, emulsions containing water, glycerol stearate, glycerin, mineral oil, synthetic spermaceti, cetyl alcohol, or combinations thereof, may be used. Preservatives may also be included in the carrier, such as one or more of butylparaben, methylparaben, propylparaben, benzyl alcohol, and ethylene diamine tetraacetate salts. The composition of the carrier can be varied so long as it does not interfere significantly with the pharmacological activity of the active ingredients of the therapeutic composition.

In one embodiment, the carrier can be a PLO gel (pluronic lecithin organogel). PLO gel contains isopropyl palmitate (a non-oleaginous emollient), soy lecithin (mixture of phospholipids), water, and Pluronic F127.

### Gelling Agents and Thickening Agents.

The compositions described herein can include one or more gelling agents to increase the viscosity of the composition. Examples of gelling agents and thickening agents, include, but are not limited to, fatty acids, fatty acid salts and esters, fatty alcohols, synthetic polymers, modified celluloses, xanthan gum, or combinations thereof. Examples of suitable synthetic polymers include polyethylene glycol (PEG), polyvinylpyrrolidone (PVP), polyvinyl alcohol (PVA), various Pluronics (poloxamers), or carbomers (e.g., Carbomer 940 or Carbomer 934). Examples of suitable modified celluloses include methylcellulose, carboxymethylcellulose (CMC), hydroxyethylcellulose (HEC), hydroxymethyl cellulose (HMC), hydroxypropyl cellulose (HPC), hydroxypropyl-methylcellulose (HPMC), or other cellulose-based gelling agents.

A variety of gelling agents is commercially available and can be obtained in many suitable molecular weights and ranges. For example, the molecular weights of the gelling agent can be about 1 kDa to about 1,000 kDa, about 10 kDa to about 1,000 kDa, about 100 kDa to about 1,000 kDa, or about 50 kDa to about 500 kDa.

Examples of thickening agents include lanolin, hard paraffin, liquid paraffin, white petrolatum, soft yellow paraffin or soft white paraffin, white beeswax, yellow beeswax, propolis (propoleum), cetostearyl alcohol, cetyl alcohol, dimethicones, emulsifying waxes, microcrystalline wax, oleyl alcohol and stearyl alcohol.

A gelling agent or thickening agent can be present in a formulation at about 0.05 wt. % to about 20 wt. %, typically about 0.1 wt. % to about 10 wt. %, about 0.1 wt. % to about 5 wt. %, about 0.5 wt. % to about 2 wt. %, about 0.8 wt. % to about 2 wt. %, or about 1-1.5 wt. %. One or more gelling agents or thickening agents may be included in a single formulation. Such agents can be employed with liquid carriers to form spreadable gels, pastes, ointments, soaps, and the like, for application directly to the skin of the user.

### Solutions and Dispersions.

Solutions of an active or its salts can be prepared in water, optionally mixed with a nontoxic surfactant. Dispersions can be prepared in glycerol, liquid polyethylene glycols, triacetin, or in a pharmaceutically acceptable oil. Under ordinary conditions of storage and use, preparations may contain a preservative to prevent the growth of microorganisms.

Pharmaceutical dosage forms can include sterile aqueous solutions or dispersions comprising the active ingredient adapted for the extemporaneous preparation of sterile solutions or dispersions, optionally encapsulated in liposomes. The ultimate dosage form should be fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, emu oil, nontoxic glyceryl esters, and suitable mixtures thereof. The proper fluidity of the composition can be maintained, for example, by the formation of liposomes, by the maintenance of the particle size in the case of dispersions, or by the use of surfactants. The prevention of the action of microorganisms can be brought about by various antibacterial and antifungal agents, for example, parabens, chlorobutanol, phenol, sorbic acid, thiomersal, and the like. In many cases, it will be preferable to include isotonic agents, for example, sugars, buffers, or sodium chloride. Prolonged absorption of the compositions can be brought about by agents delaying absorption, for example, aluminum monostearate and/or gelatin.

Solutions can be prepared by incorporating the active in a desired amount in the appropriate solvent or oil with various other ingredients enumerated herein, as desired, followed by optional filter sterilization. For powders used in the preparation of solutions, methods of preparation can include vacuum drying and freeze drying techniques, which yield a powder of the active plus any additional desired ingredient present in the prepared solutions.

### Gels.

Gels are clear, sticky, jelly-like semisolids or solids prepared from high molecular weight polymers in an aqueous or alcoholic base. Alcoholic gels are often drying and cooling. Non-alcoholic gels are more lubricating. Gels or jellies can be produced using a suitable gelling agent including, but not limited to, gelatin, tragacanth, a carbomer, or a cellulose derivative and may include glycerol as a humectant, an emollient, and/or a preservative. In some embodiments, gel formulations will include the same or similar ingredients as a solution or dispersion, with the addition of a gelling agent.

The gel can include a nonionic copolymer gelling agent. In one embodiment, the gelling agent is a nonionic polyoxyethylene-polyoxypropylene copolymer gel, for example, a Pluronic gel such as Pluronic F-127 (BASF Corp.), to provide a pluronic gel-based formulation. This gel can be advantageous because it is a liquid at low temperatures but rapidly sets at physiological temperatures, which confines the release of the agent to the site of application or immediately adjacent that site. Other formulations can be carboxymethylcellulose (CMC)-based formulations, hydroxymethyl cellulose (HMC)-based formulations, hydroxypropyl cellulose (HPC)-based formulations, or hydroxypropylmethylcellulose (HPMC)-based formulations, and the like.

### Creams.

Creams are viscous liquids or semisolid emulsions, either oil-in-water or water-in-oil. Cream bases are water-washable, and comprise an oil phase, an emulsifier, and an aqueous-phase. Water-in-oil creams may be formulated by using a suitable emulsifying agent with properties similar, but not limited, to those of the fatty alcohols such as cetyl alcohol or cetostearyl alcohol and to emulsifying wax. Oil-in-water creams may be formulated using an emulsifying agent such as cetomacrogol emulsifying wax. Suitable properties include the ability to modify the viscosity of the emulsion and both physical and chemical stability over a wide range of pH. The water soluble or miscible cream base may contain a preservative system and may also be buffered to maintain an acceptable physiological pH.

The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant (a substance, such as glycerin, sorbitol, or urea, that absorbs or helps another substance retain moisture).

The emulsifier in a cream formulation is generally a nonionic, anionic, cationic, or amphoteric surfactant. Examples of emulsifiers include, but are not limited to, fatty alcohol polyoxyethylene ether (Peregal A-20), stearates such as polyoxylstearate (Softener SG), glyceryl stearate and pegylated forms of glyceryl stearate such as PEG-5 glyceryl stearate, cetyl alcohol, dithranol, or a combination thereof.

Oil-phase ingredients can include, but are not limited to, dimethicone, dimethiconol, cyclomethicone, diisopropyl adipate, cetyl alcohol, stearyl alcohol, paraffin, petrolatum, almond oil, stearic acid, or a combination thereof. In particular aspects, aqueous ingredients can include, but are not limited to, purified water, glycerol (glycerin), propylene glycol, ethyl paraben, a humectant, or a combination thereof.

In some embodiments, the cream further comprises one or more film formers including but not limiting to polyglycerylmethacrylate, acrylates/C₁₀-C₃₀ alkyl acrylate cross-polymers; antioxidant including but not limiting to tocopheryl acetate; preservatives including but not limiting to phenoxyethanol, benzyl alcohol; other additives including but not limiting to dicaprylyl ether, disodium EDTA, sodium hydroxide, and lactic acid.

In one embodiment, the cream can include purified water, polyglycerylmethacrylate, propylene glycol, petrolatum, dicaprylyl ether, PEG-5 glyceryl stearate, glycerin, dimethicone, dimethiconol, cetyl alcohol, sweet almond oil, acrylates/C₁₀-C₃₀ alkyl acrylate cross-polymers, tocopheryl acetate, phenoxyethanol, benzyl alcohol, disodium EDTA, sodium hydroxide, lactic acid, or any combination thereof.

In another embodiment, the cream can include glycerol, light liquid paraffin, soft white paraffin, dimethicone, squalane, methyl hydroxybenzoate, dichlorobenzyl alcohol, or any combination thereof.

### Ointments.

Ointments are semisolid preparations that include the active incorporated into a fatty, waxy, or synthetic base. Ointments are typically based on petrolatum or other petroleum derivatives. The specific ointment base to be used, as will be appreciated by those skilled in the art, is one that will provide for suitable drug delivery and other desired characteristics such as emolliency or the like. As with other carriers or vehicles, an ointment base is typically inert, stable, non-irritating and non-sensitizing.

Ointment bases may be generally grouped in four classes: oleaginous bases; emulsifiable bases; emulsion bases; and water-soluble bases. Oleaginous ointment bases can include, for example, vegetable oils, fats obtained from animals such as emu oil, and semisolid hydrocarbons obtained from petroleum. Emulsifiable ointment bases, also known as absorbent ointment bases, contain little or no water and can include, for example, hydroxystearin sulfate, anhydrous lanolin, and hydrophilic petrolatum. Emulsion ointment bases are either water-in-oil (W/O) emulsions or oil-in-water (O/W) emulsions, and the oil components can include, for example, cetyl alcohol, glyceryl monostearate, lanolin, and stearic acid. Water-soluble ointment bases can be prepared from polyethylene glycols of varying molecular weight.

Lotions. Lotions are liquid or semiliquid preparations in which solid particles, including the active agent(s), are present in a water or alcohol base. Lotions are usually suspensions of solids, and can include a liquid oily emulsion of the oil-in-water type. Lotions are often desirable formulations because of the ease of applying a more fluid composition. It is generally advantageous for the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, or the like.

### Pastes.

Pastes are semisolid dosage forms in which the active agent is suspended in a suitable base. Depending on the nature of the base, pastes are divided between fatty pastes or those made from a single-phase aqueous gel. The base in a fatty paste is generally petrolatum, hydrophilic petrolatum, or the like. The pastes made from single-phase aqueous gels generally incorporate carboxymethylcellulose or the like as a base.

### Foams

Foam preparations may be formulated to be delivered from a pressurized aerosol canister, via a suitable applicator, using inert propellants. Suitable excipients for the formulation of the foam base include, but are not limited to, propylene glycol, emulsifying wax, cetyl alcohol, and glyceryl stearate. Potential preservatives include methylparaben and propylparaben.

Accordingly, the composition described herein may be formulated for any desired form of topical or transdermal administration, including slow or delayed release preparations. Formulations may include known antioxidants (e.g., vitamin E); buffering agents; lubricants (e.g., synthetic or natural beeswax); sunscreens (e.g., para-aminobenzoic acid); and cosmetic agents (e.g., coloring agents, fragrances, essential oils, moisturizers, or drying agents).

An auxiliary agent such as casein, gelatin, albumin, or sodium alginate may also be included in various formulations. Adjuvants such as fragrances and additional antimicrobial agents can be added to optimize the properties for a given use. Examples of fragrances include Ylang-Ylang oil, lavender oil, powder scent, jasmine, gardenia oil, or green tea oil. In addition, substances such as wetting or emulsifying agents, stabilizing agents, or pH buffering agents, may also be included. When a water-based carrier is used, the composition is typically near a neutral pH (+/− about 1, or 2, pH units).

Further examples of dermatological ingredients and compositions for delivering active agents to the skin are known to the art; for example, see U.S. Pat. No. 4,992,478 (Geria), U.S. Pat. No. 4,820,508 (Wortzman), U.S. Pat. No. 4,608,392 (Jacquet et al.), and U.S. Pat. No. 4,559,157 (Smith et al.). Such dermatological compositions can be used in combinations with the actives described herein in place of other actives.

The ketoalkylpyridinium compounds, e.g., with aryl substitutions, may be conveniently administered in unit dosage form; for example, containing 5 to 1000 mg, conveniently 10 to 750 mg, or conveniently 50 to 500 mg of active ingredient per unit dosage form.

A suitable dose may be in the range of from about 0.5 to about 100 mg/kg, e.g., from about 10 to about 75 mg/kg of body weight, such as 3 to about 50 mg per kilogram body weight, for example in the range of 6 to 90 mg/kg, e.g., in the range of 15 to 60 mg/kg.

### Delivery Vehicles

Delivery vehicles include, for example, microparticles, nanoparticles, liposomes and other lipid-containing complexes, and other macromolecular complexes capable of mediating delivery of a compound to a host cell. Delivery vehicles can also comprise other components or functionalities that provide beneficial properties. Such other components include, for example, components that influence binding or targeting to cells (including components that mediate cell-type or tissue-specific binding) or components that influence uptake of the vehicle by the cell. Such components can be provided as a natural feature of the vehicle or vehicles can be modified to provide such functionalities.

Vehicles within the scope of the disclosure include, but are not limited to, nanoparticles or microparticles including liposomes, e.g., neutral or cationic liposomes, such as DOSPA/DOPE, DOGS/DOPE or DMRIE/DOPE liposomes, and/or associated with other molecules. Delivery vehicles may be administered via any route including local administration, e.g., topical, subdermal, or subcutaneous administration.

### Exemplary Particle Formulations

The one or more ketoalkylpyridinium compounds, e.g., with aryl substitutions, may be present in nanoparticles or microparticles.

In one embodiment, the particles are biodegradable particles that may include or may be formed from biodegradable polymeric molecules which may include, but are not limited to polylactic acid (PLA), polyglycolic acid (PGA), co-polymers of PLA and PGA (i.e., polyactic-co-glycolic acid (PLGA)), poly-ε-caprolactone (PCL), polyethylene glycol (PEG), poly(3-hydroxybutyrate), poly(p-dioxanone), polypropylene fumarate, poly(orthoesters), polyol/diketene acetals addition polymers, poly-alkyl-cyano-acrylates (PAC), poly(sebacic anhydride) (PSA), poly(carboxybiscarboxyphenoxyphenoxy hexone (PCPP) poly[bis(p-carboxypheonoxy)methane](PCPM), copolymers of PSA, PCPP and PCPM, poly(amino acids), poly(pseudo amino acids), polyphosphazenes, derivatives of poly[(dichloro)phosphazenes] and poly[(organo)phosphazenes], poly-hydroxybutyric acid, or S-caproic acid, elastin, or gelatin. (See, e.g., Kumari et al., Colloids and Surfaces B: Biointerfaces 75 (2010) 1-18; and U.S. Pat. Nos. 6,913,767; 6,884,435; 6,565,777; 6,534,092; 6,528,087; 6,379,704; 6,309,569; 6,264,987; 6,210,707; 6,090,925; 6,022,564; 5,981,719; 5,871,747; 5,723,269; 5,603,960; and 5,578,709; and U.S. Published Application No. 2007/0081972; and International Application Publication Nos. WO 2012/115806; and WO 2012/054425; the contents of which are incorporated herein by reference in their entireties).

The biodegradable particles may be prepared by methods known in the art. The size of the particles (e.g., mean effective diameter) may be assessed by known methods in the art, which may include but are not limited to transmission electron microscopy (TEM), scanning electron microscopy (SEM), Atomic Force Microscopy (AFM), Photon Correlation Spectroscopy (PCS), Nanoparticle Surface Area Monitor (NSAM), Condensation Particle Counter (CPC), Differential Mobility Analyzer (DMA), Scanning Mobility Particle Sizer (SMPS), Nanoparticle Tracking Analysis (NTA), X-Ray Diffraction (XRD), Aerosol Time of Flight Mass Spectroscopy (ATFMS), and Aerosol Particle Mass Analyzer (APM).

In one embodiment, a particle comprises polymers including but not limited to poly(lactic-co-glycolic acid) (PLGA), polylactic acid (PLA), linear and/or branched PEI with differing molecular weights (e.g., 2, 22 and 25 kDa), dendrimers such as polyamidoamine (PAMAM) and polymethoacrylates; lipids including but not limited to cationic liposomes, cationic emulsions, DOTAP, DOTMA, DMRIE, DOSPA, distearoylphosphatidylcholine (DSPC), DOPE, or DC-cholesterol; peptide based vectors including but not limited to Poly-L-lysine or protamine; or poly(β-amino ester), chitosan, PEI-polyethylene glycol, PEI-mannose-dextrose, DOTAP-cholesterol or RNAiMAX.

In one embodiment, the delivery vehicle may be a glycopolymer-based delivery vehicle, poly(glycoamidoamine) s (PGAAs), that have the ability to complex with various polynucleotide types and form nanoparticles. These materials are created by polymerizing the methylester or lactone derivatives of various carbohydrates (D-glucarate (D), meso-galactarate (G), D-mannarate (M), and L-tartarate (T)) with a series of oligoethyleneamine monomers (containing between 1-4 ethylenamines (Liu and Reineke, 2006). A subset composed of these carbohydrates and four ethyleneamines in the polymer repeat units yielded exceptional delivery efficiency.

In one embodiment, the delivery vehicle may comprise polyethyleneimine (PEI), Polyamidoamine (PAMAM), PEI-PEG, PEI-PEG-mannose, dextran-PEI, OVA conjugate, PLGA microparticles, or PLGA microparticles coated with PAMAM, or any combination thereof. The disclosed cationic polymer may include, but are not limited to, polyamidoamine (PAMAM) dendrimers. Polyamidoamine dendrimers suitable for preparing the presently disclosed nanoparticles may include 3rd-, 4th-, 5th-, or at least 6th-generation dendrimers.

In one embodiment, the delivery vehicle may comprise a lipid, e.g., N-[1-(2,3-dioleoyloxy)propel]-N,N,N-trimethylammonium (DOTMA), 2,3-dioleyloxy-N-[2-spermine carboxamide] ethyl-N,N-dimethyl-1-propanammonium trifluoracetate (DOSPA, Lipofectamine); 1,2-dioleoyl-3-trimethylammonium-propane (DOTAP); N-[1-(2,3-dimyristloxy) propyl]; N,N-dimethyl-N-(2-hydroxyethyl) ammonium bromide (DMRIE), 3-β-[N—(N,N′-dimethylaminoethane) carbamoyl] cholesterol (DC-Chol); dioctadecyl amidoglyceryl spermine (DOGS, Transfectam); or imethyldioctadeclyammonium bromide (DDAB). The positively charged hydrophilic head group of cationic lipids usually consists of monoamine such as tertiary and quaternary amines, polyamine, amidinium, or guanidinium group. A series of pyridinium lipids have been developed (Zhu et al., 2008; van der Woude et al., 1997; Ilies et al., 2004). In addition to pyridinium cationic lipids, other types of heterocyclic head group include imidazole, piperizine and amino acid. The main function of cationic head groups is to condense negatively charged nucleic acids by means of electrostatic interaction to slightly positively charged nanoparticles, leading to enhanced cellular uptake and endosomal escape.

Lipids having two linear fatty acid chains, such as DOTMA, DOTAP and SAINT-2, or DODAC, may be employed as a delivery vehicle, as well as tetraalkyl lipid chain surfactant, the dimer of N,N-dioleyl-N,N-dimethylammonium chloride (DODAC). All the trans-orientated lipids regardless of their hydrophobic chain lengths (C_{16:1}, C_{18:1} and C_{20:1}) appear to enhance the transfection efficiency compared with their cis-orientated counterparts.

The structures of polymers useful as a delivery vehicle include but are not limited to linear polymers such as chitosan and linear poly(ethyleneimine), branched polymers such as branch poly(ethyleneimine) (PEI), circle-like polymers such as cyclodextrin, network (crosslinked) type polymers such as crosslinked poly(amino acid) (PAA), and dendrimers. Dendrimers consist of a central core molecule, from which several highly branched arms ‘grow’ to form a tree-like structure with a manner of symmetry or asymmetry. Examples of dendrimers include polyamidoamine (PAMAM) and polypropylenimine (PPI) dendrimers.

DOPE and cholesterol are commonly used neutral co-lipids for preparing liposomes. Branched PEI-cholesterol water-soluble lipopolymer conjugates self-assemble into cationic micelles. Pluronic (poloxamer), a non-ionic polymer and SP1017, which is the combination of Pluronics L61 and F127, may also be used.

In some embodiments, a biocompatible polymeric material is derived from a biodegradable polymeric such as collagen, e.g., hydroxylated collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, N-isopropylacrylamide copolymers, poly(ethylene oxide)/poly(propylene oxide) block copolymers, poly(ethylene glycol)/poly(D,L-lactide-co-glycolide) block copolymers, polyglycolide, polylactides (PLLA or PDLA), poly(caprolactone) (PCL), or poly(dioxanone) (PPS).

In another embodiment, the biocompatible material includes polyethyleneterephalate, polytetrafluoroethylene, copolymer of polyethylene oxide and polypropylene oxide, a combination of polyglycolic acid and polyhydroxyalkanoate, gelatin, alginate, poly-3-hydroxybutyrate, poly-4-hydroxybutyrate, and polyhydroxyoctanoate, and polyacrylonitrilepolyvinylchlorides.

In one embodiment, the following polymers may be employed, e.g., natural polymers such as starch, chitin, glycosaminoglycans, e.g., hyaluronic acid, dermatan sulfate and chrondrotin sulfate, and microbial polyesters, e.g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), pol (D,L-lactide-coglycolide), poly(lactic acid colysine) and polycaprolactone.

In one embodiment, the biocompatible material is derived from isolated extracellular matrix (ECM). ECM may be isolated from endothelial layers of various cell populations, tissues and/or organs, e.g., any organ or tissue source including the dermis of the skin, liver, alimentary, respiratory, intestinal, urinary or genital tracks of a warm-blooded vertebrate. ECM employed in the disclosure may be from a combination of sources. Isolated ECM may be prepared as a sheet, in particulate form, gel form and the like.

A biocompatible scaffold polymer may comprise silk, elastin, chitin, chitosan, poly(d-hydroxy acid), poly(anhydrides), or poly(orthoesters). More particularly, the biocompatible polymer may be formed polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis(p-carboxypheonoxy)methane] (PCPM), copolymers of SA, CPP and CPM, poly(amino acids), poly(pseudo amino acids), polyphosphazenes, derivatives of poly[(dichloro)phosphazenes] or poly[(organo) phosphazenes], poly-hydroxybutyric acid, or S-caproic acid, polylactide-co-glycolide, polylactic acid, polyethylene glycol, cellulose, oxidized cellulose, alginate, gelatin or derivatives thereof.

Thus, the polymer may be formed of any of a wide range of materials including polymers, including naturally occurring polymers, synthetic polymers, or a combination thereof. In one embodiment, the scaffold comprises biodegradable polymers. In one embodiment, a naturally occurring biodegradable polymer may be modified to provide for a synthetic biodegradable polymer derived from the naturally occurring polymer. In one embodiment, the polymer is a poly(lactic acid) (“PLA”) or poly(lactic-co-glycolic acid) (“PLGA”). In one embodiment, the scaffold polymer includes but is not limited to alginate, chitosan, poly(2-hydroxyethylmethacrylate), xyloglucan, co-polymers of 2-methacryloyloxyethyl phosphorylcholine, poly(vinyl alcohol), silicone, hydrophobic polyesters and hydrophilic polyester, poly(lactide-co-glycolide), N-isoproylacrylamide copolymers, poly(ethylene oxide)/poly(propylene oxide), polylactic acid, poly(orthoesters), polyanhydrides, polyurethanes, copolymers of 2-hydroxyethylmethacrylate and sodium methacrylate, phosphorylcholine, cyclodextrins, polysulfone and polyvinylpyrrolidine, starch, poly-D,L-lactic acid-para-dioxanone-polyethylene glycol block copolymer, polypropylene, poly(ethylene terephthalate), poly(tetrafluoroethylene), poly-epsilon-caprolactone, or crosslinked chitosan.

In one embodiment, the microparticles have a diameter of about 1 to about 100 microns. In one embodiment, the diameter is about 1 to about 15 microns. In one embodiment, the diameter is about 5 to about 10 microns. In one embodiment, the diameter is about 15 to about 50 microns. In one embodiment, the diameter is about 20 to about 50 microns. In one embodiment, the diameter is about 100 to about 150 microns. In one embodiment, the diameter is about 500 to about 750 microns. In one embodiment, the diameter is about 150 to about 500 microns. In one embodiment, the diameter is about 200 to about 500 microns.

In one embodiment, the nanoparticles have a diameter of about 1 to about 100 nm. In one embodiment, the diameter is about 1 to about 15 nm. In one embodiment, the diameter is about 5 to about 10 nm. In one embodiment, the diameter is about 15 to about 50 nm. In one embodiment, the diameter is about 20 to about 50 nm. In one embodiment, the diameter is about 100 to about 150 nm. In one embodiment, the diameter is about 500 to about 750 nm. In one embodiment, the diameter is about 150 to about 500 nm. In one embodiment, the diameter is about 200 to about 500 nm.

### Exemplary Methods and Delivery Vehicles

The ketoalkylpyridinium compounds, e.g., with aryl substitutions, are useful to prevent, inhibit or treat microbial, e.g., fungal, mold, yeast and bacterial, e.g., gram-negative or gram-positive, bacterial, infections. In one embodiment, the ketoalkylpyridinium compounds, e.g., with aryl substitutions, are useful to prevent, inhibit or treat biofilms. Biofilms are microbial populations or communities massed together and surrounded by an extracellular matrix that forms a protective layer. Numerous microbes form biofilms, e.g., Trichophyton rubrum, other dermatophytes, and non-dermatophyte molds (NDMs) have been shown to form biofilms including but not limited to Aspergillus and Candida species.

The disclosure thus provides for a method to prevent, inhibit or treat a fungal or bacterial infection in an avian or a mammal, comprising: administering to the avian or mammal an effective amount of a composition comprising one or more of the disclosed compounds. In one embodiment, the composition is topically administered. In one embodiment, the mammal is a human. In one embodiment, the mammal is not a human. In one embodiment, the amount is fungicidal. In one embodiment, the amount inhibits biofilm activity. In one embodiment, the composition is administered to the skin. In one embodiment, the composition is administered to the eye. In one embodiment, the avian or mammal has keratitis. In one embodiment, the avian or mammal is infected with Aspergillus, Candida, Fusarium, or Pseudomonas.

Also provided is a method to disinfect a medical device, comprising: contacting the device and a composition comprising one or more the disclosed compounds. In one embodiment, the device is a catheter. In one embodiment, the device is a cannula. In one embodiment, the device is a needle. In one embodiment, the device is a prosthetic joint.

In one embodiment, a wound covering, e.g., bandage, comprising one or more of the compounds is applied to an avian or mammal in need thereof. In one embodiment, the wound covering comprises a dressing formed of one or more synthetic or natural materials. In one embodiment the wound covering is an adhesive bandage.

The composition may be administered to an acute wound or chronic wound, or a site with an acute or chronic infection, e.g., infected with Aspergillus, Candida or Fusarium.

In one embodiment, the delivery vehicle for the ketoalkylpyridinium compounds, e.g., with aryl substitutions, is a polymeric scaffold, e.g., formed of collagen or a derivative thereof or cellulose or a derivative thereof.

In one embodiment, the delivery vehicle for the ketoalkylpyridinium compounds, e.g., with aryl substitutions, is an organic polymeric, e.g., a polymeric nanoparticle, nanoemulsion or micelle. Polymer nanoparticles may be produced either in the form of gel-like particles or solid polymer particles. Nano- or microgel particles are prepared from swollen hydrophilic polymers; they are generally obtained by ionotropic gelation, a one-step process in which a counter-ion is added to a polyelectrolytic polymer, thus producing micro- or nanoparticles though ionic interactions. In contrast, solid polymer particles are based on organo-soluble, poorly hydrophilic polymers such as PLGA or PLA, and they may be produced by methods such as oil-in-water or water-in-oil-in-water emulsion evaporation. In one embodiment, the delivery vehicle comprises carboxymethyl chitosan (CMC) nanoparticles (NPs), poly(ethyl acrylate-co-methacrylic acid) (MAA) microgels, hyaluronic acid nanogels PLGA NPs, micelles, e.g., methoxy poly(ethylene glycol)-b-poly(glutamic acid) (mPEG-b-PGlu), nanostructured lipid carriers (NLC) or liposomes.

In one embodiment, the delivery vehicle comprises inorganic NPs, e.g., metallic NPs such as gold (AuNPs) or silver (AgNPs) NPs.

In one embodiment, the delivery vehicle comprises carbon nanotubes, graphene, fullerene or carbon quantum dots (CQDs).

### Subjects

The subject may be any animal, including a human and non-human animal. Non-human animals include all vertebrates, e.g., mammals and non-mammals, such as non-human primates, sheep, dogs, cats, cows, horses, chickens, amphibians, and reptiles, although mammals are envisioned as subjects, such as non-human primates, sheep, dogs, cats, cows and horses. The subject may also be livestock such as, cattle, swine, sheep, poultry, and horses, or pets, such as dogs and cats.

In one embodiment, subjects include human subjects suffering from or at risk for the medical diseases and disorders described herein, e.g., infections. The subject is generally diagnosed with the condition by skilled artisans, such as a medical practitioner.

The methods described herein can be employed for subjects of any species, gender, age, ethnic population, or genotype. Accordingly, the term subject includes males and females, and it includes elderly, elderly-to-adult transition age subjects, adults, adult-to-pre-adult transition age subjects, and pre-adults, including adolescents, children, and infants.

Examples of human ethnic populations include Caucasians, Asians, Hispanics, Africans, African Americans, Native Americans, Semites, and Pacific Islanders. The methods may be more appropriate for some ethnic populations such as Caucasians, especially northern European populations, as well as Asian populations.

The term subject also includes subjects of any genotype or phenotype as long as they are in need of treatment, as described above. In addition, the subject can have the genotype or phenotype for any hair color, eye color, skin color or any combination thereof. The term subject includes a subject of any body height, body weight, or any organ or body part size or shape.

The invention will be further described by the following non-limiting examples.

### Example I

### Materials and Methods

### Strains, Media, Reagents and Instrumentation.

All yeast strains were maintained on YPD from 25% glycerol stocks stored at −80° C. All A. fumigatus strains were maintained on glucose minimal media (GMM; from 25% glycerol stocks stored at −80° C. A. fumigatus and A. terreus CFF clinical isolates and F. oxysporum were received from Dr. Robert Cramer (Dartmouth College), SPF98 was received from Dr. W. Scott Moye-Rowley (University of Iowa), C. neoformans DUMC clinical isolate series were received from Dr. John Perfect (Duke University). S. aureus and P. aeruginosa strains were received from Dr. Dominique Limoli (University of Iowa). TWO #15 and TWO #17 were received from Dr. Ted White (University of Missouri Kansas City). Reagents were purchased as at least reagent grade from Aldrich, Acros or Alfa Aesar unless otherwise specified and used without further purification. Solvents were purchased from Fischer Scientific (Pittsburgh, PA) and were either used as purchased or redistilled with an appropriate drying agent. Compounds used for structure-activity studies were synthesized according to methods described below, and all the compounds were identified to be least 95% pure using HPLC. Analytical TLC was performed using precoated Silica G TLC Plates, w/UV254, aluminum backed, purchased from Sorbtech (Norcross, GA) and visualized using UV light. Flash chromatography was carried out using with a Biotage Isolera One (Charlotte, NC) system using the specified solvent. Microwave reactions were performed using Biotage Initiator+(Charlotte, NC). Purity analysis were performed on an Agilent 1100 HPLC utilizing a C-18 column (Waters Nova-Pak; 3.9×100 mm) with the following method: Solvent A 306=H₂O (0.1% TFA), Solvent B=Acetonitrile; 0 to 20 minutes, (10 to 90% B), 20 to 25 minutes (90 to 10% B); detection was set at two wavelengths (245 and 280 nm). Purity of all final compounds was above 95%. All final compounds were analyzed by high resolution MS (HRMS) using a Bruker Maxis Plus Quadrupole Time-of-Flight (QTOF or QqTOF). 1H and 13C NMR were recorded on either a BrukerAvance III outfitted with a 5 mm BBFO Z-gradient probe or a Avance III instrument is equipped with a 5 mm BBFO probe. The chemical shifts are expressed in parts per million (ppm) using suitable deuterated NMR solvents.

### Dichlorodiphenylmethane (Wiederhold, 2022).

To a round flask was weighed benzophenone (1 eq) and PCl₅ (1.5 eq). The reaction was refluxed for 2 hours at 150° C. After 2 hours, the reaction was cooled, 40 mL of DCM was added to the resultant solution followed by transferring to 250 mL Erlenmeyer flask. The resultant solution was cooled to 0° C. and 40 mL of distilled water was added. The resultant solution was transferred to a separatory funnel, the DCM layer was separated, washed twice with 40 ml distilled water, and dried with anhydrous sodium sulfate. The DCM solvent was evaporated in rotary evaporator to give corresponding dichloride. The crude product was immediately used for the next step without purification.

### 2-chloro-1-(2,2-diphenylbenzo[d][1,3]dioxol-5-yl)ethenone (2)

To a round flask was weighed dichlorodiphenylmethane (1 g, 4.2 mmol) and 2-chloro-1-(3,4-dihydroxyphenyl) ethanone (0.79 g, 4.2 mmol) under nitrogen. The reaction was refluxed at 180° C. for 30 minutes under nitrogen. After 30 minutes. The crude product was transferred to silica gel chromatography with linear gradient of 2-10% (EtOAc/Hexane). The fractions corresponding to the desired product were collected and solvent evaporated using rotatory evaporator to give yellow viscous liquid as the desired product which turned to a solid upon dryness. Yield; 0.7 g, 47%, ¹H NMR (500 MHz, CDCl₃) δ 7.69 (m, 4H), 7.61-7.57 (m, 2H), 7.44 (m, 6H), 6.98 (d, J=332 7.9 Hz, 1H), 4.63 (s, 2H). ¹³C NMR (75 MHz, CDCl₃), 189.26, 152.05, 148.05, 139.46, 129.56, 129.01, 128.41, 125.14, 118.58, 108.52, 108.38, 45.84.

### 4-acetamido-1-(2-(2,2-diphenylbenzo[d][1,3]dioxol-5-yl)-2-oxoethyl)pyridin-1-ium chloride (Taff et al., 2013)

To a round flask containing 2-chloro-1-(2,2-diphenylbenzo[d][1,3]dioxol-5-yl)ethenone (0.34 0.97 mmol) dissolved in acetonitrile (10 mL) was weighed the 4-aminopyridine (0.09 g, 0.97 338 mmol). The reaction was refluxed for 2 hours at 100° C. in which precipitate was formed. After 2 hours, the solvent was evaporated using rotatory evaporator and the gummy solid precipitated with 10% Hexane in DCM, filtered and dried to give a powdered solid product in form of the chloride salt. HPLC purity; 98%. Yield; 0.38 g, 88%, ¹H NMR (300 MHz, DMSO) δ 12.12 (s, 1H), 8.65 (d, J=342 6.9 Hz, 2H), 8.22 (d, J=6.9 Hz, 2H), 7.74 (d, J=8.3 Hz, 2H), 7.68 (s, 1H), 7.57-7.55 (m, 4H), 7.48-7.32 (m 6H), 7.32 (d, J=8.2 Hz, 1H), 6.21 (s, 2H), 2.27 (s, 3H). ¹³C NMR (75 MHz, DMSO), 344 189.88, 171.71, 152.87, 151.87, 147.59, 146.99, 139.30, 130.21, 129.18, 129.00, 126.25, 125.70, 345 118.36, 114.77, 109.56, 108.65, 64.75, 25.06, HRMS for [M+H]⁺ calculated; 451.1652, found; 451.1658.

### Planktonic Growth Phase Antifungal Activity Assays.

Minimum inhibitory concentrations were determined using CLSI guidelines (Beattie & Krysan, 2021; Wiederhold, 2021). All yeasts were cultured overnight in 3 mL YPD at 30° C., then washed twice in sterile PBS. Two-fold serial dilutions of each compound were prepared in RMPI+MOPS pH 7 (Gibco RPMI 1640 with L-glutamine [11875-093] and 0.165M 352 MOPS), then 1×10³ cells were added per well. Plates were incubated at 37° C. for 24 hours (C. albicans and S. cerevisiae) or 72 hours (C. neoformans). For A. fumigatus and F. oxysporum wells were inoculated with 1.25×10⁴ conidia. Plates were incubated at 37° C. for 48 hours for A. fumigatus and 72 hours for F. oxysporum. All MICs are defined as the well with complete growth inhibition as determined by visual inspection with the exception of MICs of fluconazole against C. albicans where the MIC is defined as at least 50% reduction in growth compared to the control.

### Biofilm Phase Antifungal Activity Assays.

These assays were performed as previously described (Mao et al., 2020; Ramage et al., 2001). C. albicans and C. auris, overnight cultures were washed in sterile PBS and adjusted to 1×10⁶ CFU/mL in RPMI+MOPS pH 7. 100 μL cells were added to each well and incubated at 37° C. for 24 hours. A two-fold dilution series of the compounds was prepared in RPMI+MOPS with equal vehicle concentrations. Media was removed and biofilms were gently washed with PBS, then 200 μL of each drug dilution was added to wells and plates were incubated at 37° C. for an additional 24 hours. Media was removed and biofilms were gently washed with PBS, then 100 μL of XTT solution (0.83 mg/mL XTT sodium salt [Sigma, Cat #X4626]+32 μg/mL PMS [Sigma, Cat #P9625] in PBS) was added to each well. Plates were incubated at 37° C. for 30 minutes, then the absorbance was measured at 490 nm.

### Red Blood Cell Lysis Assay.

Hemolysis assays were performed with defibrinated sheep's blood (Lampire, Cat #7239001). Blood was washed three times with PBS then resuspended to about 50% hematocrit in PBS. A two-fold dilution series of the compounds in 200 μL PBS with equal DMSO concentration across the series, then red blood cells were added with a final concentration of 2% hematocrit. Cells were incubated at RT for 2 hours in a v-bottom microtiter plate, then plates were spun down and supernatant was transferred to a flat bottom microtiter plate for absorbance measurement at 570 nm. Compounds were tested in technical triplicate in at least two independent assays performed on different days.

### Mammalian Cell Culture Toxicity Assay.

HepG2 cells (ATCC, #) were maintained and cultured in HepG2 media (DMEM (Gibco, Cat #11965-092) with 5% FBS, 20 mM Glutamine, and Penicillin/Streptomycin) at 37° C. with 5% CO₂. Cells were seeded in 96-well plates at a density of 1.25×10⁴ cells/well and incubated overnight at 37° C. with 5% CO₂, then media was removed and replaced with media containing a two-fold dilution series of compounds with equal DMSO concentrations across all wells including no compound controls. Cells were incubated for an additional 24 hours. Supernatant was removed and used to quantify lactate dehydrogenase release using the CyQuant LDH assay kit (Invitrogen, Cat #C20300) following the manufacturer's directions. LDH signal was normalized to a max lysis control. The remaining cells were then used to quantify cellular metabolism by XTT. Briefly, cells were washed, then 0.9 mg/mL XTT (Sigma, CAT #X4626)+320 μg/mL PMS (Phenothiazine methosulfate; Sigma, Cat #P9625) in HepG2 media was added to each well. Plates were incubated at 37° C., with 5% CO₂ for 2 hours, then absorbance was measured at 490 nm.

Rat Model of Candida albicans Vascular Catheter Infection.

C. albicans biofilm growth during infection of implanted medical devices was measured using an external jugular-vein, rat-catheter infection model (Nett et al., 2012). Briefly, a 1×10⁶ cells/ml inoculum for each strain or strain combination was allowed to grow on an internal jugular catheter placed in a pathogen-free female rat (16-week old, 400 g) for 24 hours. After this period, the catheter volumes were removed and the catheters were flushed with 0.9% NaCl. The biofilms were dislodged by sonication and vortexing. Viable cell counts were determined by dilution plating. Three replicates were performed for each strain.

### Scanning Electron Microscopy of Catheter Biofilms.

After a 24 hours biofilm formation phase, the devices were removed, sectioned to expose the intraluminal surface, and processed for SEM imaging. Briefly, one milliliter fixative (4% formaldehyde and 1% glutaraldehyde in PBS) was added to each catheter tube and tubes were fixed at 4° C. overnight. Catheters were then washed with PBS prior to incubation in 1% OsO4 for 30 minutes. Samples were then serially dehydrated in ethanol (30-100%). Critical point drying was used to completely dehydrate the samples prior to palladium-gold coating. Samples were imaged on a SEM LEO 1530, with Adobe Photoshop 2022 (v. 23.2.2) used for image compilation.

Porcine Skin Model of Candida auris Colonization.

The collection of porcine skin samples was conducted under protocols approved by the University of Wisconsin-Madison Institutional Animal Care and Use Committee in accordance with published National Institutes of Health (NIH) and United States Department of Agriculture (USDA) guidelines. Excised skin was cleaned and shaved as described previously (Johnson et al., 2022). Full-thickness samples were obtained by 12 mm punch and placed in 12-well plates containing 3 mL Dulbecco's Modified Eagle Medium (DMEM) (Lonza, Walkersville, MD, USA) supplemented with 10% fetal bovine serum (FBS) (Atlanta biologicals, Lawrenceville, GA, USA), penicillin (1,000 U/mL), and streptomycin (1 mg/mL) (Corning, Manassas, VA, USA) for 6 hours. Tissues were rinsed in DBPS and moved to semi-solid media (6:4 ratio of 1% agarose (BIO-RAD, Hercules, CA, USA) in DPBS:DMEM with 10% FBS). Paraffin wax was applied around the edge of the skin and 10 μL C. auris suspended in synthetic sweat medium at 10⁷ cells/mL was applied. Samples were incubated at 24 hours and compound A (8 μg/mL or 16 μg/mL) in synthetic sweat medium was applied. After 24 hours a sterile swab was used to remove compound from skin surface. Samples were vortexed in DPBS and plated on YPD+chloramphenicol to assess viable burden.

### Results

Aryl Methyl Keto-N-Alkylated Pyridinium Molecules have Broad Spectrum Antifungal Activity.

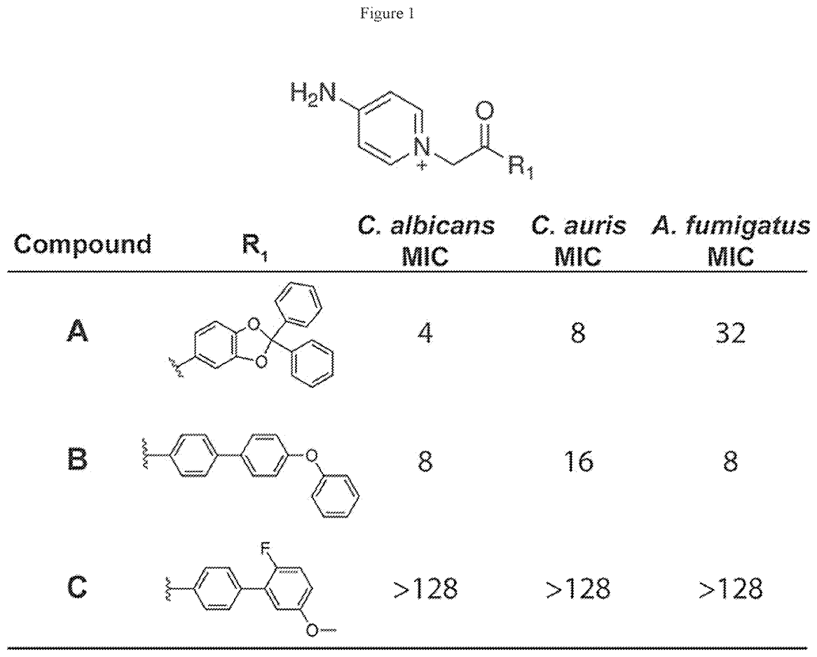

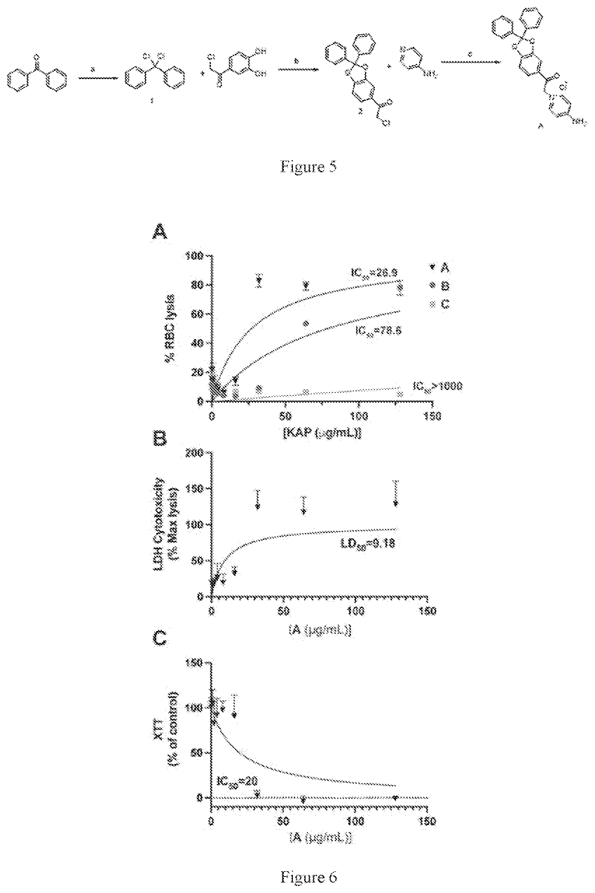

A set of small molecule libraries were screened for compounds against Aspergillus fumigatus using recently developed high throughput screening assay applicable for this filamentous mold (Beattie & Krysan, 2021). A molecule with the aryl methyl keto-N-alkylated pyridinium (keto-alkyl pyridinium, KAP) scaffold (FIG. 1A) was identified a hit in the screen. A set of analogs of this initial hit were prepared and tested, leading to two derivatives with similar activity profiles (compounds A and B, FIG. 1A); compound C was used a control as it is inactive but has a very similar chemical structure compared to the active derivatives. Although >30 KAP derivatives were prepared, no clear structure-activity relationships (SAR) could be identified to explain the variation in antifungal properties of this series (see FIG. 5 for general synthesis approach). Pyridinium cations are widely believed to target membranes as part of their mechanism of action and a non-protein target could explain this difficult-to-interpret SAR (Edlind et al., 2005; Mao et al., 2020). Although other potential targets have also been proposed, a biochemically or genetically confirmed alternative target to the membrane has not been reported (Shen et al., 2020; Simm et al., 2022). It is, however, interesting that small changes in substitution patterns in comparing compounds A/B to compound C (FIG. 1A) lead to dramatic changes in antifungal activity without clearly identifiable changes in physicochemical properties. These observations suggest that the molecules have structurally specific interactions with a target and imply that interaction with membranes is not solely due to their amphipathic properties.

The scope of the antifungal activity for compounds A and B was explored by CLSI methods for a range of fungal pathogens including yeasts and molds (Table 2). In general, A/B are more consistently active against yeasts (C. albicans, C. neoformans, C. auris) compared to molds (A. fumigatus, A. terreus, and F. oxysporum). Although the MIC for some isolates of A. fumigatus was similar to those for yeast, significant strain-to-strain variability in activity toward Aspergillus was observed. Compounds A/B were also tested against two fluconazole-resistant C. albicans strains (TWO 15/17, Mao et al., 2020) that have increased expression of efflux pumps. There is no difference in susceptibility in these strains compared to the reference strain SC5314 (Table 2). Furthermore, compounds A and B were both active against drug susceptible (0381) and multi-drug resistant (0390) C. auris isolates (Table 2).

TABLE 2

| Species | Strain | A MIC | B MIC |
|---|---|---|---|
| A. fumigatus | CEA10 | 32 | 8 |
| | AF293 | 2 | 1 |
| | SPF98 | 64 | 16 |
| | Cas^{R} | 32 | 16 |
| | CFF S014 | 8 | NT |
| | CFF S008 | 64 | NT |
| | CFF S007 | 64 | NT |
| | CFF S001 | 64 | NT |
| | CFF S029 | 4 | NT |
| | CFF S003 | 4 | NT |
| A. terreus | CFF S023 | 2-4 | NT |
| C. albicans | SC5314 | 4 | 8 |
| | TWO #15 | 4 | 4 |
| | TWO #17 | 4 | 4 |
| C. glabrata | KK2001 | 4 | 1 |
| C. auris | 0381 | 8 | 16 |
| | 0390 | 2 | 4 |
| C. neoformans | H99 | 4 | 2 |
| | DUMC 118.00 | 4 | 2 |
| | DUMC 122.01 | 4 | 4 |
| F. oxysporum | DUMC Fo | 16 | 16 |
| P. aeruginosa | PAO1 | 64 | 32 |
| S. aureus | Newman | 32 | 16 |

Since pyridinium cations such as cetylpyridinium chloride also have activity against bacteria, compounds A and B were tested against the Gram-positive bacteria, Staphylococcus aureus, and the Gram-negative bacteria, Pseudomonas aeruginosa. Compared to cetylpyridinium chloride, compounds A and B were 8-16-fold less active against S. aureus (cetylpyridinium chloride MIC 2 μg/mL, reported in Mao et al., 2020) but were more active against P. aeruginosa (cetylpyridinium chloride MIC 250 μg/mL reported in Mao et al., 2020). In contrast, the activity of A/B were within 2-fold of the MICs reported for cetylpyridinium chloride against C. albicans and pyrvinium pamoate against C. auris.

To determine if compounds interacted with clinically used antifungal drugs, checkerboard assays were performed and fractional inhibitory concentration indices were determined. Compound B showed indifferent interactions with both caspofungin and amphotericin B against C. albicans but showed a complex interaction with fluconazole (Table 3). The FICI for compound B and fluconazole is 8.25 due to the 8-fold increase in fluconazole concentration in the combination; however, the concentration of compound B at FIC is reduced by 4-fold relative to compound B as a single agent (Table 3). Interestingly, Edlind et al. also observed that cetylpyridinium chloride showed antagonism with fluconazole and induced expression of the efflux pumps CDR1 and CDR2 (Edlind et al., 2005).

TABLE 3
Fractional inhibitory concentration indices (FICIs) of KAPs with clinical antifungals.
FICIs were calculated using the MIC (μg/mL) of each drug alone (MIC_{A}) and in
combination (MIC_{C}) based on at least two independent checkboard assays performed
on separate days. Interactions are defined as: 0.5 ≥ FICI = synergy; 0.5 < FICI < 2 =
additive; 2 < FICI < 4 = indifferent; FICI ≥ 4 = antagonism.

| | Clinical | B | | Clinical | | |
|---|---|---|---|---|---|---|
| Species | antifungal | MIC_{A} | MIC_{C} | MIC_{A} | MIC_{C} | FICI |
| C. albicans | Fluconazole | 16 | 4 | 0.125 | 1 | 8.25 |
| C. albicans | Amphotericin B | 4 | 1 | 0.5 | 0.25 | 0.75 |
| A. fumigatus | Voriconazole | 16 | 2 | 0.25 | 0.125 | 0.625 |

In contrast to the antagonistic interaction of compound B with fluconazole in C. albicans, voriconazole, the gold standard therapy for aspergillosis, showed an indifferent interaction. These data indicate that the antagonistic interaction between compound B and azoles in C. albicans by compound B does not occur with A. fumigatus. Efflux pump mediated resistance to azoles is not as prevalent in A. fumigatus compared to C. albicans and, thus, the mechanisms of efflux pump induction and function are not as well defined.

### KAPs are Active Against Candida Biofilms and A. Fumigatus Hyphae

In general, pyridinium cations may be too toxic for systemic administration because many examples of this class directly lyse red blood cells (Obando et al., 2018). Consistent with those expectations, compound A and compound B cause significant red blood cell lysis (LD₅₀ A: 26.9 μg/mL; B: 78.6 μg/mL; and C>1000 μg/mL, FIG. 6) while compound C, which has no antifungal activity also had no activity against red blood cells. This observation provides implicit support for the membrane as an important target of these molecules. Both compounds A and B also showed toxicity against the human cell line HegG2 with LD₅₀ of 9 μg/mL and 20 μg/mL using cell lysis and metabolic activity assays, respectively (FIG. 6). Consequently, the KAPs, like other quaternary nitrogen anti-infectives, are likely to be suitable for topical or other non-systemic applications and not feasible for systemic therapy.

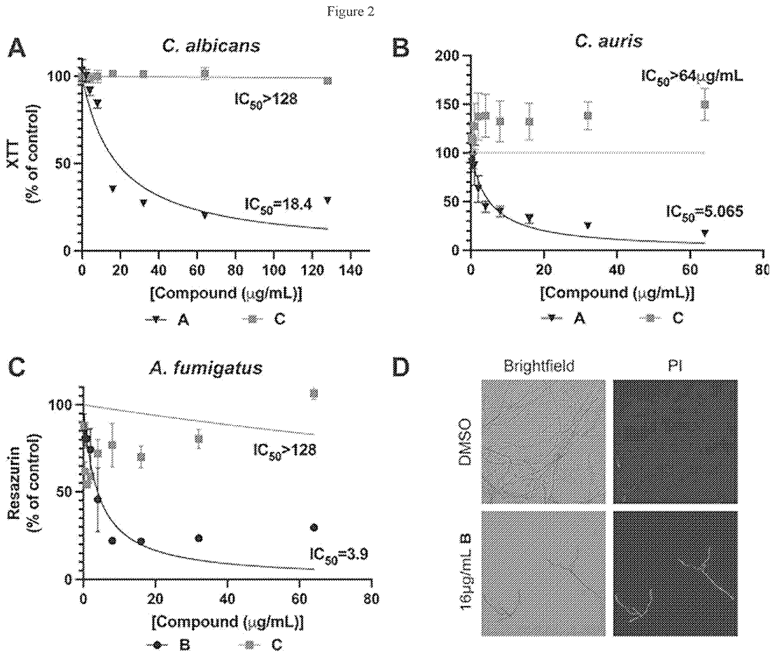

As discussed above, many anti-infectives have dramatically reduced activity against biofilm phase organisms compared to the planktonic growth phase of standard CLSI activity assays. Compound A, the most active against planktonic Candida, was tested against Candida albicans and Candida auris biofilms; Candida biofilms were established for 24 hours in the absence of drug, and then the biofilms were treated with a dilution series of the compounds for an additional 24 hours. The metabolic activity of the biofilms was then assayed using the standard XXT reduction assay (Ramage et al., 2001). Consistent with planktonic results, compound A was active (FIG. 2A, IC₅₀=18.4 μg/mL) while C was not. The biofilm activity of compound A was reduced by 4-fold relative to planktonic growth (MIC 4 μg/mL, Table 2).

CLSI testing of antifungal activity of molds is based on inoculation with conidia and, therefore, measures inhibition of germination (Widerhold, 2021). For example, voriconazole is the gold standard for treatment of pulmonary infection and inhibits germination. The most important A. fumigatus infection that requires non-systemic therapy is fungal keratitis (Hoffman et al., 2022). At the time of clinical presentation, A. fumigatus is exclusively in the hyphal form. It was, therefore, of interest to determine if KAP compounds were active against A. fumigatus hyphae; we only tested compound B because it was most active against A. fumigatus under CLSI conditions. A. fumigatus CEA10 was incubated for 24 hours prior to exposure to a dilution series of compound B or C for an additional 24 hours. The metabolic activity of the cultures was determined using resazurin as previously described (Mao et al., 2020). The IC₅₀ of compound B in this assay was 3.9 μg/mL (FIG. 2C). To further characterize the mode of action of the KAPs against A. fumigatus hyphae, CEA10 was treated with compound B for 24 hours and then the cells were stained with propidium iodide, a dye that is excluded from cells with intact membranes (FIG. 2D). Whereas cells treated with DMSO control showed essentially no staining, compound B-treated cells showed uniform uptake of dye, indicating loss of membrane integrity. Further, DIC images of hyphae treated with B show loss of refractivity and fragmentation of hyphae, consistent with hyphal killing (FIG. 2D). Taken together, these data indicate that KAPs directly kill hyphal stage A. fumigatus.

KAP a is Efficacious in an In Vivo Model of C. albicans Venous Catheter Infection.

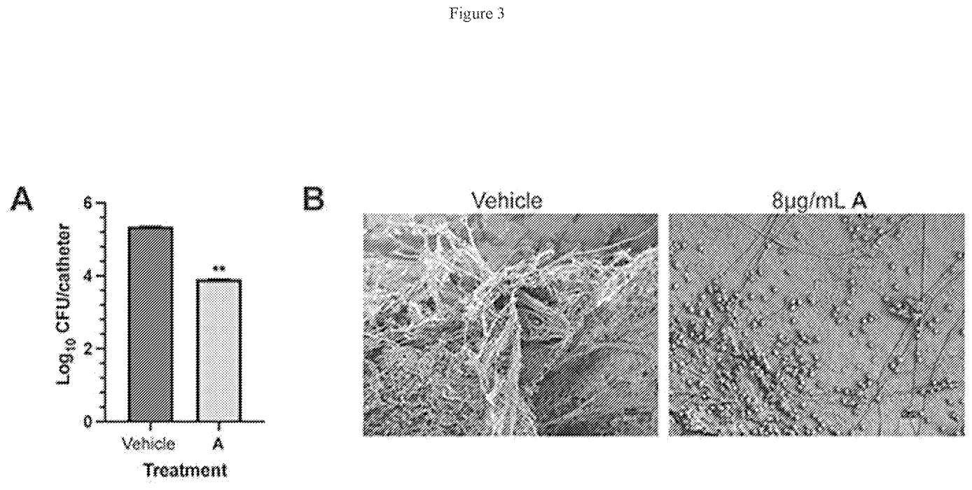

One potential approach to managing central venous catheter infections is to treat the lumen of the catheter with an anti-infective solution that is locked within the catheter and not introduced into the patient (Chandra et al., 2018). This strategy is most often applied to catheters infected with bacteria but has been proposed for fungal infections as well (Pappas et al., 2016; Mermel et al., 2009). To test the efficacy of KAPs in this setting, a rat model of C. albicans venous catheter infection was employed (Nett et al., 2023). The catheter was infected with C. albicans SC5314 and, on post-infection day 1, treated with A (8 μg/mL solution within catheter) or vehicle. After 24 hours, the catheter was removed and processed for fungal burden and scanning electron microscopy. Compound A-treated catheters showed >1 log₁₀ reduction in fungal burden relative to vehicle-treated catheters (FIG. 3A). Consistent with these results, the treated catheters showed a dramatic reduction in the extent of remaining biofilm. Thus, compound A is able to disrupt a pre-formed C. albicans biofilm in vitro and in vivo.

KAP a Reduces C. auris Colonization of Skin in an Ex Vivo Porcine Model

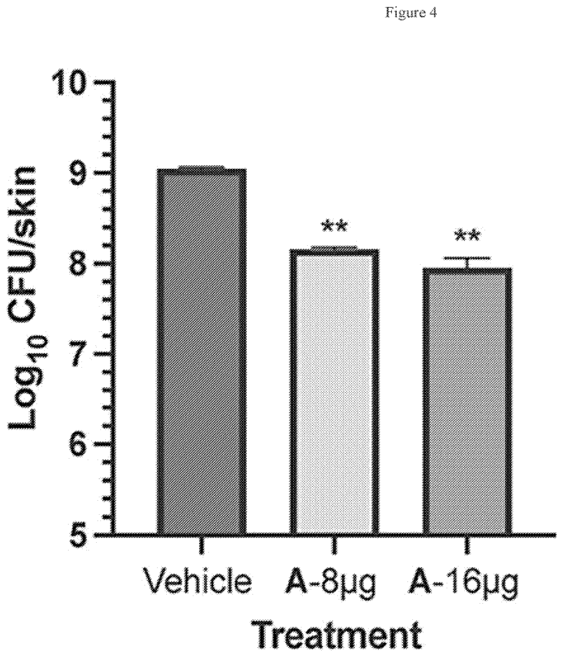

The ability of C. auris to colonize human skin is likely associated with its persistence in infected patients and its ability to transmit from person-to-person (Horton & Nett, 2020). Because compound A showed comparable activity against C. auris during planktonic and inanimate-substrate biofilm growth, we tested its activity in an ex vivo porcine model of C. auris skin colonization (Johnson et al., 2022). Consistent with these in vitro data, compound A reduced the fungal burden of the colonized porcine skin by about 1 log₁₀ at a concentration of 8 μg/mL (FIG. 4). The fungal burden was not reduced further at 16 μg/mL of compound A, suggesting that absorption/adsorption or physicochemical factors may limit efficacy. The activity of compound A was greater than that of 2% chlorhexidine (about 0.5 log₁₀ reduction, Johnson et al., 2021) and comparable to the synergistic activity of 2% chlorhexidine/70% isopropanol (1.0 log₁₀ reduction). Due to limitations in compound A availability, the data for skin treated with compound A was collected 24 hours after a single treatment while the studies with chlorhexidine and 70% isopropanol were based on a 72 hour experiment with three daily applications of study compounds. These experimental distinctions notwithstanding, compound A shows promising activity against C. auris and is likely at least as effective, and possibly more effective, than 2% chlorhexidine.

### Discussion

Although new therapeutic options for systemic therapy of fungal infections are sorely needed (Perfect et al., 2022), agents with promise in the treatment of other types of fungal infections, particularly those involving biofilm and biofilm-like growth phases, would also be quite valuable (Taff et al., 2013; Kean & Ramage, 2019). Here, the activity of a structurally series of pyridinium cation-based antifungal molecules was characterized and their potential for the treatment of non-systemic fungal infections demonstrated. Compounds A and B show comparable or improved activity relative to other cationic nitrogen-based anti-infectives/anti-septics such as chlorhexidine and cetylpyridinium chloride against fungal pathogens (Johnson et al., 2021; Edlind et al., 2005; Simm et al., 2022). In addition, compounds A and B have similar activity to the pyridinium-based drug pyrvinium pamoate against C. auris against in vivo biofilms (Simm et al., 2022), further supporting the utility of this class of molecule against this important emerging and drug-resistant pathogen.

The in vivo and ex vivo experiments provide compelling proof-of-principle data for the potential use of these scaffolds in the setting of antifungal lock therapy for intravascular catheters and as possible disinfectants for C. auris skin colonization. Although they were not compared head-to-head, compound A is more active when compared to data previously reported chlorhexidine using the same model (Johnson et al., 2021). Chlorhexidine was also shown to have activity in a mouse model of colonization (Huang et al., 2021). Since these compounds are variations on previously validated chemical structures with application to topical and other non-systemic uses, a strong premise exists for their potential development as topical antifungal drugs. The toxicity of the KAPs against human cell lines is comparable or slightly less than cetylpyridinium chloride (cetylpyridinium chloride LD₅₀: 6 μg/mL (Kim et al., 2021) v 9 and 20 μg/mL for compounds A and B respectively), a widely used topical anti-infective. Additional potential applications for which the data provide support are oropharyngeal candidiasis and fungal keratitis. The latter infection is a globally important cause of blindness for which there is no generally effective medical therapy (Hoffman et al., 2022). Compounds A and B have reasonable activity against the two most common etiologic agents of fungal keratitis: A. fumigatus and Fusarium oxysporum (Table 2). The direct activity against hypha is an important feature of this series but additional optimization will be needed to improve the consistency of activity and therapeutic index to become useful for fungal keratitis.

The mechanism of action for quaternary nitrogen-based antifungals such as the KAPs is likely to involve, at least in part, the direct disruption of membrane structures (Mao et al., 2020). Recent work by Sim et al. suggested that pyrvinium pamoate interferes with mitochondrial function (Simm et al., 2022) and mode of action studies of the cationic amidine antifungal candidate T-2307 as well as other cationic ammonium antifungals also showed that mitochondrial disruption contributes to their activity (Shen et al., 2020; Yamashita et al., 2019). A hallmark of mitochondrial disruption in C. albicans is an inability to grow on non-fermentable carbon sources such as glycerol. Indeed, the MIC of T-2307 decreases dramatically when the cells are grown on 2% glycerol compared to 2% dextrose (Yamashita et al., 2019). A change in MIC for compounds A or B was not observed when tested in the same medium with glycerol instead of glucose as carbon source, indicating that mitochondrial disruption is unlikely to be a major mode of action (data not shown). Attempts to generate resistant mutants by serial passaging were also unsuccessful which further suggests either multiple targets or non-protein targets such as the membrane are likely to contribute to the antifungal activity of the KAPs.

It is interesting that compound A that, like cetylpyridinium chloride, is antagonistic with fluconazole in C. albicans in checkerboard assays. Edlind et al. showed that cetylpyrdinium chloride induces the expression of the efflux pumps CDR1 and CDR2 but that deletion of these genes has no effect on its activity (Edlind et al., 2005). Compounds A and B are as active against reference strains as they are against fluconazole-resistant clinical strains shown to have increased expression of efflux pumps (Table 2, White, 1997). Thus, it seems that both types of pyridinium-based antifungal molecules alter expression of fluconazole pumps without being susceptible to their effects. As such, these observations imply that they have similar mechanisms of action that are best attributed to membrane disruption.

In summary, in vitro and in vivo data suggest that the KAP-type pyridinium cation molecules may be agents for the non-systemic treatment of fungal infections.

### Example 2

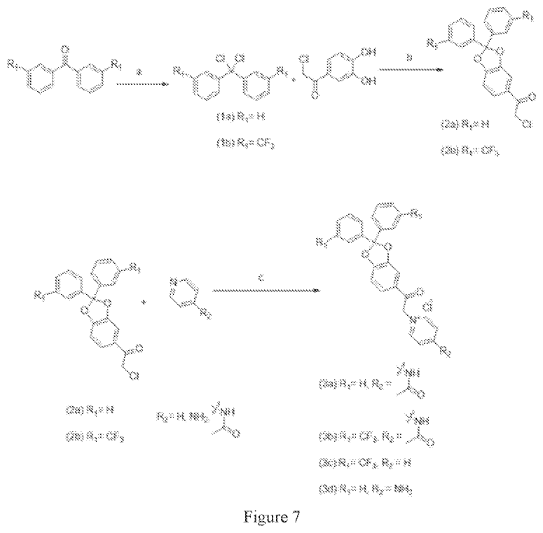

The keto-alkyl-pyridinium compounds represent a structurally novel extension of pyridinium class antiseptics that have broad spectrum antifungal activity. The structurally unique aspect is exemplified in structures 3a-d (FIG. 7). Previous pyridinium agents have alkyl substituents whereas our compounds have aryl ketone substituents. In addition, the dioxolanone ring system is unique. In addition to the structurally unique features, we have shown that these molecules are active against a broad range of medically important fungi. Finally, we have shown these molecules have activity against C. albicans biofilms in an in vivo rat catheter model and are more active than chlorhexidine against C. auris skin infection using an ex vivo model of this process.

### Synthesis of Compounds 3a-d.

The compounds were synthesized by Tim Hagen at Northern Illinois University. The synthesis is outlined in FIG. 1. These are easily generated in a three-step synthesis and were purified to >95% purity.

### In Vitro, In Vivo and Ex Vivo Antifungal Activity:

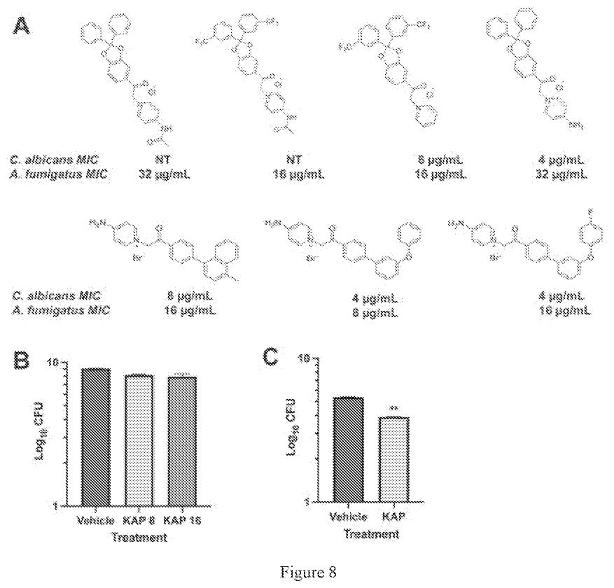

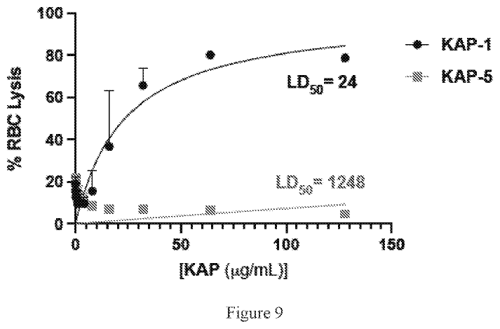

The in vitro activity for a selected set of examples is shown in FIG. 8A and represents the most active molecules we have generated. The dioxolanone derivatives have the best activity against pathogenic yeast (3a-d) while compound 4 has the best activity against molds. The activity of 3d was tested for its ability to treat intravascular catheters infected with C. albicans using a rat catheter model. As shown in FIG. 8B, treatment significantly reduced the fungal burden of the catheter in 24 hr. The activity of 3d was also tested in a C. auris skin colonization model. C. auris is a multi-drug resistant yeast that colonizes skin and is resistant to most standard antiseptics used in the hospital. As shown in FIG. 8C, treatment reduces C. auris colonization of skin by about 1 log₁₀ CFU whereas chlorhexidine gluconate is minimally active. Finally, these compounds are active against Aspergillus fumigatus, a mold, which also causes skin and eye infections (FIG. 8A).

This class of molecules has applications to the treatment of fungal infections involving mucosal, eye, and skin surfaces including thrush, oropharyngeal candidiasis, vulvovaginal candidiasis, fungal keratitis, and nail infections. The compounds also may prove effective in the treatment or prevention of fungal infections of intravascular catheters. Finally, they may also be useful for decolonizing patients who have acquired C. auris colonization.

### Example 3

Additional results are shown below in Table 4 and in FIG. 9.

TABLE 4

| | | A. | C. |
|---|---|---|---|
| | | fumi- | albi- |
| | | gatus | cans |
| | | MIC | MIC |
| | | (μg/ | (μg/ |
| Compound | R Group | mL) | mL) |
| KAP-1 | 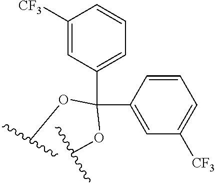 [SMILES: *OC(O*)(c1cccc(C(F)(F)F)c1)c1cccc(C(F)(F)F)c1] | 16 | 1 |
| KAP-2 | 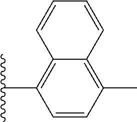 [SMILES: *c1ccc(C)c2ccccc12] | 16 | 8 |
| KAP-3 | 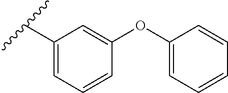 [SMILES: *c1cccc(Oc2ccccc2)c1] | 8 | 4 |
| KAP-4 | 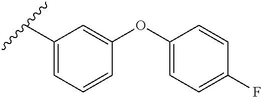 [SMILES: *c1cccc(Oc2ccc(F)cc2)c1] | 16 | 4 |
| KAP-5 | 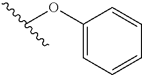 [SMILES: *Oc1ccccc1] | >64 | NT |

MICs of KAP-1 and structural analogs against A. fumigatus CEA10 or C. albicans SC5314. NT=not tested.

### REFERENCES

Beattie & Krysan, mSphere., 6 (4): e0053921 (2018).
Chandra et al., Antimicrob. Agents Chemother., 62: e00722 (2018).
Chen et al., Agents Chemother., 54:3233 (2010).
Edlind et al., Antimicrob. Agents Chemother., 49:843 (2005).
Hoffman et al., J. Fungi (Basel), 8:1067 (2022).
Horton & Nett, Curr. Clin. Microbiol. Rep., 7:51 (2020).
Huang et al., Cell Host Microbe., 29:210 (2021).
Jacobs et al., Antimicrob. Agents Chemother., 66: e0005322 (2022).
Johnson et al., J. Fungi, 7:804 (2021).
Johnson et al., Methods Mol. Biol., 2517:251 (2022).
Kean & Ramage, mSphere. 4: e00458 (2019).
Kim et al., J. Appl. Toxicol., 41:470 (2021).
Mao et al., Antimicrob. Agents Chemother., 64: e00576 (2020).
Mermel et al., Clin. Infect. Dis., 49:1 (2009).
Nett et al., Methods Mol. Biol., 845:547 (2012).
Obando et al., Chem. Med. Chem., 13:1421 (2018).
Pappas et al., Clin. Infect. Dis., 62: e1(2016).
Perfect et al., Front Cell Infect Microbiol., 12:881272 (2022).
Ramage et al., Antimicrob. Agents Chemother., 45:2475 (2001).
Rybak et al., Curr. Opin. Microbiol., 70:102208 (2022).
Shen et al., Med. Mycol., 58:521 (2020).
Simm et al., Microbiol. Spectr., 10: e0010022 (2022).
Taff et al., Future Microbiol., 8:1325 (2013).
White, Antimicrob. Agents Chemother., 41:1482 (1997).
Widerhold, Open Forum Infect. Dis., 8:444 (2021).
Wiederhold, J. Fungi (Basel), 8:857 (2022).
Yamashita et al., Antimicrob. Agents Chemother., 63 (8): e00374 (2019).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A compound having a structure according to Formula I: 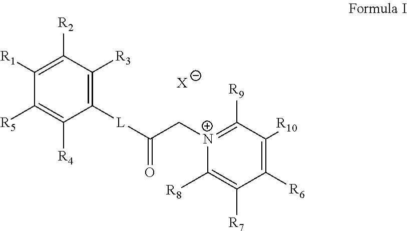
wherein
R1 and R2 are each independently H, alkyl, OH, SH, alkoxy, alkylthio, aryloxy, arylthio, alkyl, aryl, arylmethyl, arylmethoxy, F, Cl, Br, I, CF₃, CF₂H, CN, NO₂, NH₂, NH(alkyl), NH(aryl), N(alkyl)₂, NH(CO)(alkyl), or NH(CO)(aryl), or R1 and R2 taken together with the atoms to which they are attached form a 1,3-dioxolane ring and have the structure 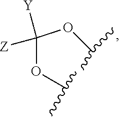
wherein Y and Z are each independently H, alkyl, or aryl;
R3 is H, alkyl, OH, SH, alkoxy, alkylthio, aryloxy, arylthio, alkyl, aryl, arylmethyl, arylmethoxy, F, Cl, Br, I, CF₃, CF₂H, CN, NO₂, NH₂, NH(alkyl), NH(aryl), N(alkyl)₂, NH(CO)(alkyl), or NH(CO)(aryl);
R4 and R5 are each independent H, alkyl, OH, SH, alkoxy, alkylthio, aryloxy, arylthio, alkyl, aryl, arylmethyl, arylmethoxy, F, Cl, Br, I, CF₃, CF₂H, CN, NO₂, NH₂, NH(alkyl), NH(aryl), N(alkyl)₂, NH(CO)(alkyl), or NH(CO)(aryl), or R4 and R5 taken together with the atoms to which they are attached form a fused aromatic ring;
L is a bond or an aromatic ring;
R6 is H, alkyl, OH, SH, alkoxy, alkylthio, aryloxy, arylthio, alkyl, aryl, arylmethyl, arylmethoxy, F, Cl, Br, I, CF₃, CF₂H, CN, NO₂, NH₂, NH(alkyl), NH(aryl), N(alkyl)₂, NH(CO)(alkyl), or NH(CO)(aryl);
R7, R8, R9, and R10 are each independently H, alkyl, OH, SH, alkoxy, alkylthio, aryloxy, arylthio, alkyl, aryl, arylmethyl, arylmethoxy, F, Cl, Br, I, CF₃, CF₂H, CN, NO₂, NH₂, NH(alkyl), NH(aryl), N(alkyl)₂, NH(CO)(alkyl), or NH(CO)(aryl); and
X is a pharmaceutically acceptable conjugate base.

2. The compound of claim 1, wherein at least one of R1 and R2 are aryloxy, or R1 and R2 taken together with the atoms to which they are attached form a 1,3-dioxolane ring and have the structure: 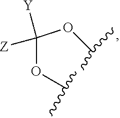
wherein Y and Z are each independently H, alkyl, or aryl.

3. (canceled)

4. The compound of claim 1, wherein L is phenylene or a bond.

5. (canceled)

6. The compound of claim 1, wherein R6 is H, NH₂, NH(alkyl), NH(aryl), N(alkyl)₂, NH(CO)(alkyl), or NH(CO)(aryl).

7. (canceled)

8. (canceled)

9. (canceled)

10. (canceled)

11. (canceled)

12. The compound of claim 1, wherein R4 and R5 taken together with the atoms to which they are attached have the structure:

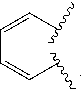 [SMILES: */C=C\C=C/*]

13. The compound of claim 1, having a structure according to Formula II:

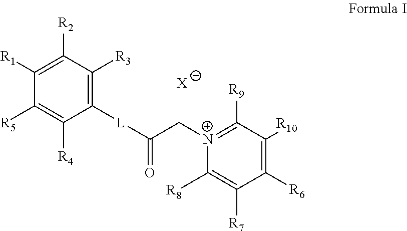

14. The compound of claim 1, having a structure according to Formula III:

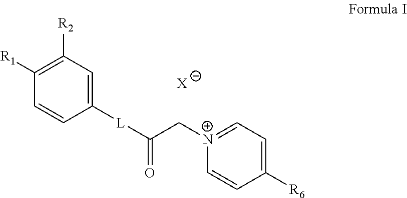

15. The compound of claim 1, having a structure according to Formula VII:

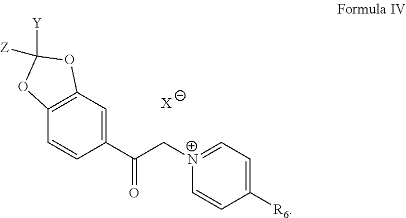

16. The compound of claim 1, having a structure according to Formula V: 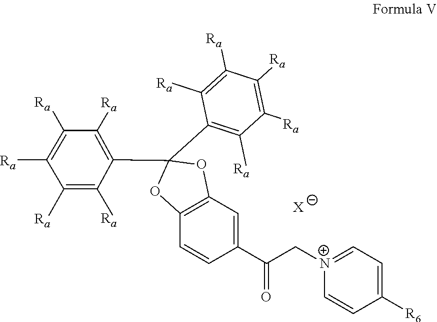
wherein Ra is H, alkyl, OH, SH, alkoxy, alkylthio, aryloxy, arylthio, alkyl, aryl, arylmethyl, arylmethoxy, F, Cl, Br, I, CF₃, CF₂H, CN, or NO₂.

17. The compound of claim 1, having a structure according to Formula VI: 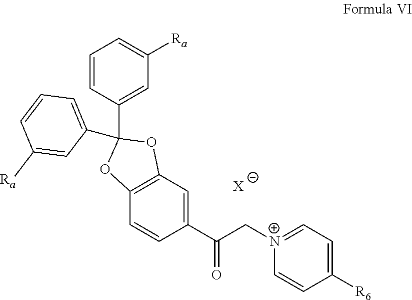
wherein Ra is H, alkyl, OH, SH, alkoxy, alkylthio, aryloxy, arylthio, alkyl, aryl, arylmethyl, arylmethoxy, F, Cl, Br, I, CF₃, CF₂H, CN, or NO₂.

18. The compound of claim 1, having a structure according to Formula VII:

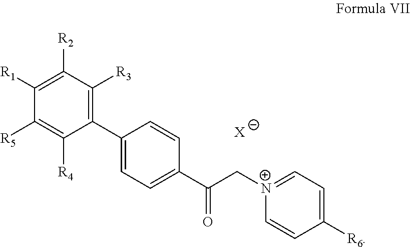

19. (canceled)

20. A compound having the structure:

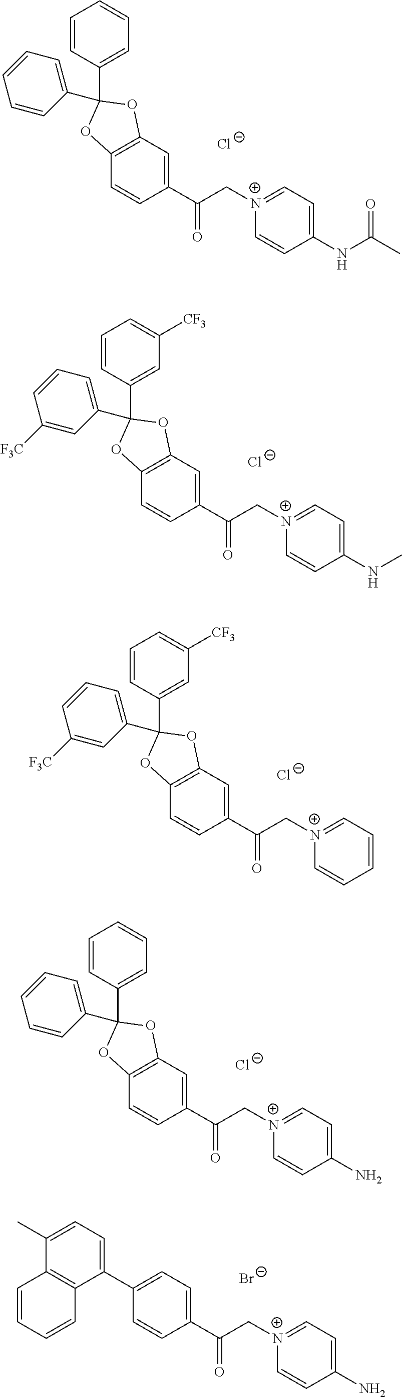

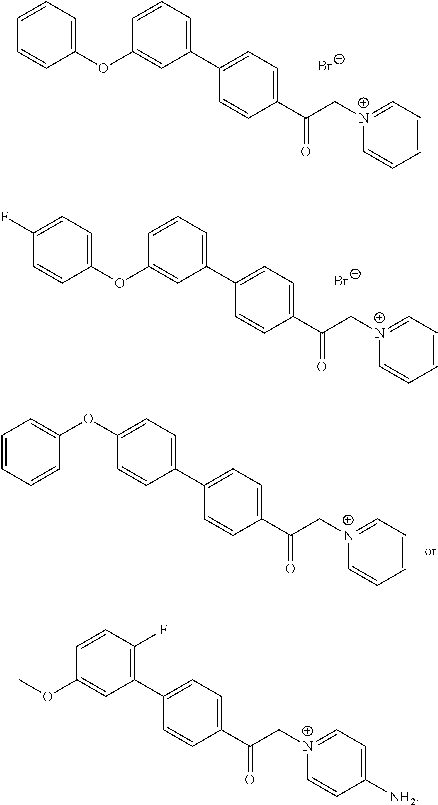

21. (canceled)

22. A method to prevent, inhibit or treat a fungal or bacterial infection in an avian or a mammal, comprising: administering to the avian or mammal an effective amount of a composition comprising the compound of claim 1.

23. The method of claim 22, wherein the composition is topically administered.

24.-25. (canceled)

26. The method of claim 22, wherein the amount is fungicidal or wherein the amount inhibits biofilm activity.

27. (canceled)

28. The method of claim 22, wherein the composition is administered to the skin, teeth, eye or oral cavity.

29.-32. (canceled)

33. A method to disinfect a medical device, comprising: contacting the device and a composition comprising the compound of claim 1.

34. The method of claim 33, wherein the device is a catheter, a cannula, a needle, a prosthetic joint or dentures.

35.-37. (canceled)

38. A wound covering comprising the compound of claim 1 and optionally comprising an adhesive.

39. The wound covering of claim 38 which comprises a dressing formed of one or more natural materials or one or more synthetic materials.

40.-41. (canceled)
